(19) United States
(12) Patent Application Publication
(10) Pub. No.: US 2021/0017582 A1
(43) Pub. Date: Jan. 21, 2021

(54) DETECTION OF GENOMIC SEQUENCES AND PROBE MOLECULES THEREFOR

(71) Applicant: Safeguard Biosystems Holdings Ltd., London (GB)

(72) Inventors: Holger Klapproth, Freiburg (DE); Sonja Bednar, Gundelfingen (DE)

(73) Assignee: Safeguard Biosystems Holdings Ltd., London (GB)

(21) Appl. No.: 16/839,339

(22) Filed: Apr. 3, 2020

Related U.S. Application Data
(60) Provisional application No. 62/876,413, filed on Jul. 19, 2019.

(51) Int. Cl. C12Q 1/689; C12Q 1/6806

(52) U.S. Cl. CPC C12Q 1/689; C12Q 1/6806

(57) ABSTRACT

Methods of identifying homologous genomic sequences that may be present in a sample utilizing virtual probes, arrays for distinguishing homologous genomic sequences, systems for distinguishing homologous genomic sequences, and probe molecules useful in the methods, arrays, and systems of the disclosure.

### 1. CROSS-REFERENCE TO RELATED APPLICATIONS

This application claims the priority benefit of U.S. provisional application No. 62/876,413, filed Jul. 17, 2019, the contents of which are incorporated herein in their entireties by reference thereto.

### 2. SEQUENCE LISTING

The instant application contains a Sequence Listing which has been submitted electronically in ASCII format and is hereby incorporated by reference in its entirety. Said ASCII copy, created on Jan. 25, 2020, is named SGB-006US_ST25 and is 1,541 bytes in size.

### 3. BACKGROUND

Identification of infectious disease agents is often critical in determining the proper diagnosis of and course of treatment for an individual. Misidentification of an infectious agent may result in an improper or ineffective course of treatment. Improvements in sequencing technologies have aided in determining the sequences of the genomes, in part or in whole, of a number of disease causing bacteria. Closely related bacterial species often comprise genomic sequences that share a very high degree of sequence identity. Distinguishing between closely related species requires sensitive and accurate methods of detecting differences between two or more homologous genomic sequences. Distinguishing between homologous genomic sequences using a single oligonucleotide probe molecule can in some instances be impractical, if not impossible.

Thus, there is a need for new methods for distinguishing closely related species of microorganisms.

### 4. SUMMARY OF THE INVENTION

The present disclosure provides methods of identifying genomic sequences and/or distinguishing homologous genomic sequences that may be present in a sample. The methods utilize combinations of probe molecules that cannot individually but can collectively distinguish homologous genomic sequences from closely related species of microorganisms and may also identify genomic sequences that are likely to be present in a sample. Such combinations of probe molecules are referred to herein as “virtual probes” for convenience.

Virtual probes can comprise a plurality (e.g., two, three, or more than three) of individual probe molecules. The hybridization of a genomic DNA (or a PCR product amplified from the genomic DNA) to an individual probe molecule may not be sufficient, without sequencing, to differentiate the genomic DNA from a homologous genomic DNA of a genetically related species that may be present in the same sample, particularly when using universal primers that target conserved sequences in related species to amplify the genomic DNA. However, when the genomic DNA or corresponding PCR amplicon is probed with a virtual probe comprising two or more probe molecules, the difference in hybridization patterns to the virtual probes can differentiate the genomic DNA from two related species or homologous amplicons amplified therefrom.

Accordingly, by virtue of comprising a plurality of probe molecules (e.g., probe molecules with distinguishable signals), the virtual probes of the disclosure can in combination distinguish a genomic sequence from a homologous genomic sequence (or amplicons prepared therefrom) and identify the microbial species present in a biological sample. For example, in accordance with the methods of the disclosure, a combination of two or three oligonucleotide probe molecules can in combination form a virtual probe that distinguishes between amplicons from related species such as S. mitis and S. pneumoniae. Thus, when a sample is used as a source of template DNA, for example in a PCR reaction, the hybridization of any resulting PCR product to the virtual probe can determine which of the two species is present in a sample.

The methods of identifying homologous genomic sequences using virtual probes disclosed herein were developed following the realization that species-specific oligonucleotide probe molecules designed using the sequences from bacterial 16S rRNA genes showed cross reactivity among different species. Because of low variability among the sequences of this region of the genome, species specific probe molecules could not be designed. However, it was discovered that different species could nonetheless be distinguished by analyzing the signals from hybridization to a virtual probe that combines multiple oligonucleotide probe molecules that are by themselves individually incapable of distinguishing between different species.

In one aspect, the present disclosure provides methods of determining whether a first organism (or corresponding first genome) and/or a second organism (or corresponding second genome) is present in a sample.

Such methods can comprise probing the sample with a virtual probe for the first organism and the second organism to determine the presence or absence of one or more target nucleic acids corresponding to the first genome or second genome. The target nucleic acids can be, for example, genome fragments or amplicons produced in a DNA amplification reaction such as PCR. The virtual probe comprises two or more probe molecules, each of which is capable of specifically hybridizing to one or more of the target nucleic acids corresponding to the first genome and/or one or more homologous target nucleic acids corresponding to the second genome. Because the probe molecules hybridize non-identically to the target nucleic acids corresponding to the first and second genomes, the virtual probe can distinguish between the target nucleic acids corresponding to the first genome and the target nucleic acids corresponding to the second genome.

An exemplary method comprises the steps of:
(a) Performing a polymerase chain reaction (PCR) amplification reaction on the sample using one or more pairs of PCR primers capable of hybridizing to, and initiating PCR amplification from, the first and second genome if present in the sample. Each set of primers gives rise to an amplicon set that is preferably unique to each organism that might be present in the sample. Thus, amplification results in a first amplicon set if the first genome is present and a second, different amplicon set if the second genome is present in the sample. If only a single pair of PCR primers is used, each amplicon set contains only a single amplicon, and when a plurality of PCR primer pairs is used, an amplicon set can contain two or more amplicons (e.g., a plurality of single amplicons).
(b) Following step (a), any resulting PCR amplification products are probed with a virtual probe to determine the presence or absence of the first amplicon set and second amplicon set. Because the virtual probe comprises two or more probe molecules (e.g., two or more oligonucleotide probe molecules) capable of specifically hybridizing to the first amplicon set and second amplicon set in a distinct manner, the virtual probe can distinguish between the first amplicon set and the second amplicon set. The probe molecules within each virtual probe can be distinguished by virtue of having different labels (e.g., fluorescent labels, for example molecular beacons labeled with different fluorescent labels) or being positioned at discrete locations on an array.

Accordingly, hybridization of the PCR amplification products of the PCR reaction to the virtual probe can distinguish between the first and second genomes, thereby identifying the presence of the first and/or second organisms in the sample. As used herein, the reference to the presence of an organism in the sample does not mean that the sample had a live organism, merely that sufficient genomic DNA from the organism was present in the sample to be detected or to serve as a template for an amplification reaction such as a PCR reaction. Likewise, the reference to the presence of a genome in a sample does not mean that the sample had an intact genome, merely that sufficient DNA from the genome was present in the sample to be detected or to serve as a template for an amplification reaction such as a PCR reaction.

The first amplicon set and the second amplicon set can each comprise one amplicon (referred to as a “first amplicon” and a “second amplicon,” respectively), for example when a single set of primers is used in a PCR amplification reaction. Alternatively, the first amplicon set and/or the second amplicon set can comprise more than one amplicon (each amplicon in the first amplicon set referred to as a “first amplicon” and each amplicon in the second amplicon set referred to as a “second amplicon”), for example when more than one set of primers is used in a PCR amplification reaction. Further exemplary methods for distinguishing homologous amplicons from homologous genomic sequences are described in Section 6.2 and numbered embodiments 1 to 86, 130 to 132, and 135, infra.

The disclosure further provides arrays for distinguishing homologous genomic sequences, systems for distinguishing homologous genomic sequences, and oligonucleotide probe molecules which are useful, for example, in the methods, arrays, and systems of the disclosure.

In one aspect, the present disclosure provides addressable arrays for distinguishing a first genomic sequence from a first genome and a second, homologous genomic sequence from a second genome. The addressable arrays of the disclosure can be used, for example, in the methods described herein. An addressable array of the disclosure can comprise a group of positionally addressable oligonucleotide probe molecules, each at a discrete location on the array, where each probe molecule in the group of oligonucleotide probe molecules comprises a nucleotide sequence that is 90% to 100% complementary to 15 to 40 consecutive nucleotides in the first genomic sequence or second genomic sequence. The addressable array may further optionally comprise one or more control probe molecules.

Exemplary addressable arrays of the disclosure are described in Section 6.3 and numbered embodiments 87 to 129 and 153 to 155, infra.

In another aspect, the present disclosure provides systems for distinguishing between a first genomic sequence and a second, homologous genomic sequence if present in a sample. An exemplary system can comprise:
(a) an optical reader for generating signal data for each probe molecule location of an array of the disclosure; and
(b) at least one processor which:
   (i) is configured to receive signal data from the optical reader;
   (ii) is configured to analyze the signal data for one or more virtual probes (e.g., a virtual probe having features as described herein); and
   (iii) has an interface to a storage or display device or network for outputting a result of the analysis.

Exemplary systems are described in Section 6.4 and numbered embodiments 133 to 134, infra.

In another aspect, the present disclosure provides exemplary oligonucleotide probe molecules suitable for use in a virtual probe and kits comprising two or more of such oligonucleotide probe molecules. The oligonucleotide probe molecules of the disclosure can be included on an addressable array of the disclosure and/or used in the methods of the disclosure. Exemplary oligonucleotide probe molecules and virtual probes are described in Section 6.2.4 and numbered embodiments 136 to 152, infra. Exemplary kits are described in Section 6.5 and numbered embodiments 156 to 167, infra.

### 5. BRIEF DESCRIPTION OF THE FIGURES

FIG. 1A-1B is a dendrogram (split between FIG. 1A and FIG. 1B) prepared from the 16S rRNA genes of several Staphylococcus species obtained from GenBank. The tree was constructed using the CLC sequence viewer software with bootstrap analysis to show the confidence in the data. Each species of the Staphylococcus group is represented with two sequences which are shown by Gen Bank accession numbers. The bootstrap values at each node of the tree (in whole numbers) define the percentage of confidence in the data across the replicates. The higher the bootstrap value the more supportive is the data for respective taxa. The horizontal lines for a respective species are called branches and represent the amount of genetic change in that species. The unit of branch length is shown as the number of changes or substitutions divided by the length of the sequence.

FIG. 2A-2B represents a dendrogram (split between FIG. 2A and FIG. 2B) of species from the Streptococcus viridians group created from multiple alignment of 16s rRNA and 16s-23s rRNA genome sequences. I—Streptococcus bovis group, II—Streptococcus anginosus group, III—Streptococcus salivarius group, IV—Streptococcus mitis group, and V—Streptococcus mutans group. The numbers represent bootstrap values which indicate the percentage of confidence in the dataset. Depending on the alignment patterns and their pathogenic importance, the species are addressed as three groups—I, II and III.

FIG. 3 is a diagram of 16S rRNA and 16S-23S rRNA ITS genomic sequences, and 16S forward (16S Fw), 16S reverse (16S Rv), ITS forward (ITS Fw), and ITS reverse (ITS Rv) primers.

FIG. 4 illustrates a primer pair useful for the asymmetric PCR methods described in Section 6.2.3.4, comprising an Unextended Primer (which can be a traditional primer used in symmetric PCR processes) and an Extended Primer composed of an “A” region, which is complementary to the target nucleic acid, a “B” region which includes a Direct Repeat or Inverted Repeat of at least a portion of the “A” region, and an optional “C” region, which can include a spacer sequence and/or part of all of a restriction endonuclease recognition site.

FIG. 5A-5C. FIG. 5A illustrates intermolecular hybridization of Extended Primers that occurs when the “B” region contains an Inverted Repeat of at least a portion of the “A” region. FIG. 5B illustrates intermolecular hybridization of Extended Primers that occurs when the “B” region contains a Direct Repeat of at least a portion of the “A” region. FIG. 5C illustrates intramolecular hybridization of Extended Primers that occurs when the “B” region contains an Inverted Repeat of at least a portion of the “A” region. Preferably, the region of complementarity between the “A” region and the “B” region is at or near the 5′ end of the “A” region.

FIG. 6 illustrates the denaturation step in an asymmetric PCR reaction described in Section 6.2.3.4. In the denaturation step a PCR reaction mixture (typically containing a biological sample containing or at risk of containing target nucleic acid, an Asymmetric Primer Pair, a thermostable DNA polymerase, and PCR Reagents) is heated to above the melting point of the target nucleic acid, resulting in denaturation of the target nucleic acid (if present) and the Extended Primer in the Asymmetric Primer Pair so as to form single strands.

FIG. 7 illustrates the annealing step of the exponential phase of the asymmetric PCR reactions described in Section 6.2.3.4, which occurs below the melting temperature of the Unextended Primer. Both the Unextended Primer and Extended Primer in the Asymmetric Primer Pair hybridize to their respective complementary strands. FIG. 7 shows annealing (also referred to as hybridization or binding) to target DNA, as occurs in the initial cycles of PCR, but in subsequent cycles annealing is likely to occur between primers and complementary sequences in PCR products. Because of the “B” and optional “C” regions in the Extended Primer, the PCR products will have those sequences or their complements, as depicted in FIG. 8B and FIG. 9.

FIGS. 8A-8B: FIG. 8A and FIG. 8B illustrate the extension step of the exponential phase of the asymmetric PCR reactions described in Section 6.2.3.4, during which the thermostable DNA polymerase extends the primer DNA using the complementary DNA as template. The region of extension is depicted in dashed lines. The template in FIG. 8A is a strand of target DNA, and in FIG. 8B is a strand of PCR product produced using the Asymmetric Primer Pair and the target DNA.

FIG. 9 illustrates the simultaneous annealing and extension step of the linear phase of the asymmetric PCR reactions described in Section 6.2.3.4, which occurs above the melting temperature of the Unextended Primer and below the melting temperature of the Extended Primer, using the PCR Product Strand 2 as template. This results in asymmetric amplification of PCR Product Strand 2, resulting in an excess of PCR Product Strand 2 molecules relative to PCR Product Strand 1 molecules by the end of the PCR reaction.

FIG. 10A-B illustrate how two (FIG. 10A) or three (FIG. 10B) probe molecules can be used in a virtual probe for coagulase negative Staphylococcus (CNS). The signals from the hybridization of a PCR amplification product to the two or three probe molecules can be combined using Boolean operators to determine if a CNS is present in a sample.

FIG. 11A-B show signals for various oligonucleotide probe molecules when bound to 16S rRNA amplicons from Streptococcus mitis (FIG. 11A) and Streptococcus pneumoniae (FIG. 11B).

FIG. 12 shows signal intensities for various oligonucleotide probe molecules when bound to PCR amplicons from Streptococcus pneumoniae, Streptococcus mitis and Streptococcus oralis.

FIG. 13 shows signal intensities for various oligonucleotide probe molecules when bound to PCR amplicons from Salmonella enterica and Escherichia coli.

FIG. 14 shows signal intensities for various oligonucleotide probe molecules when bound to PCR amplicons from Klebsiella pneumoniae and Klebsiella oxytoca.

FIG. 15 shows signal intensities for various oligonucleotide probe molecules when bound to PCR amplicons from Enterobacter cloacae, Enterobacter asburiae, and Enterobacter hormaechei.

### 6. DETAILED DESCRIPTION

### 6.1. Definitions

Amplicon: An amplicon is a nucleic acid molecule produced by a PCR amplification reaction.

Asymmetric Primer Pair: A Primer Pair consisting of an Extended Primer and an Unextended Primer.

Corresponding: In relation to two nucleic acid strands of different length that share sequence identity or complementary, the term “corresponding” refers to the region of sequence overlap or complementarity present in both strands, as the context dictates.

Direct Repeat: In the context of the “B” region of an Extended Primer, “Direct Repeat” means a nucleotide sequence that is the direct complement to a portion of the “A” region (i.e., has the complementary sequence in the same 5′ to 3′ order).

Extended Primer: A PCR primer that contains (a) an “A” region at its 3′ end that has at least 75% sequence identity to a corresponding region Target Strand 1 or at least 75% sequence complementarity to a corresponding region in Target Strand 2; (b) a “B” region at its 5′ end that comprises a sequence that is complementary to at least a portion of the “A” region; and (c) an optional “C” region positioned between the “A” and “B” regions.

Homologous genomic sequence: Homologous genomic sequences are genomic sequences found in different species or strains which have shared ancestry but which are not identical in nucleotide sequence. Exemplary homologous genomic sequences include 16S rRNA genes, 23S rRNA genes, and 16S-23S internal transcribed spacer region (ITS) sequences.

Inverted Repeat: In the context of the “B” region of an Extended Primer, “Inverted Repeat” means a nucleotide sequence that is the reverse complement to a portion of the “A” region (i.e., has the complementary sequence in the opposite 5′ to 3′ order).

Melting temperature (Tₘ): the temperature at which a one half of a DNA duplex will dissociate to become single stranded. The Tₘ's of linear primers comprised of deoxyribonucleotides (DNA) have been commonly calculated by the “percent GC” method (PCR PROTOCOLS, a Guide to Methods and Applications, Innis et al. eds., Academic Press (San Diego, Calif. (USA) 1990) or the “2 (A+T) plus 4 (G+C)” method (Wallace et al., 1979, Nucleic Acids Res. 6 (11):3543-3557) or the “Nearest Neighbor” method (Santa Lucia, 1998, Proc. Natl. Acad. Sci. USA 95: 1460-1465; Allawi and Santa Lucia, 1997, Biochem. 36:10581-10594). For the purpose of the claims, the Tₘ of a DNA is calculated according to the “Nearest Neighbor” method, and non-naturally occurring bases (e.g., 2-deoxyinosine) are treated as adenines.

PCR Product Strand 1: PCR Product Strand 1 refers to the strand in a double-stranded PCR product produced from target nucleic acid and an Asymmetric Primer Pair which is complementary to the Unextended Primer of the Asymmetric Primer Pair.

PCR Product Strand 2: PCR Product Strand 1 refers to the strand in a double-stranded PCR product produced from target nucleic acid and an Asymmetric Primer Pair which is complementary to the Extended Primer of the Asymmetric Primer Pair.

PCR Reagents: unless the context dictates otherwise, the term “PCR Reagents” refers to components of a PCR reaction other than template nucleic acid, thermostable polymerase and primers. PCR Reagents typically include dNTPs (and may include labeled, e.g., fluorescently labeled, dNTPs in addition to unlabeled dNTPs), buffers, and salts containing divalent cations (e.g., MgCl₂).

Primer: A DNA oligonucleotide of at least 12 nucleotides that has a free hydroxyl group at its 3′ terminus. Primers can include naturally and non-naturally occurring nucleotides (e.g., nucleotides containing universal bases such as 3-nitropyrrole, 5-nitroindole or 2-deoxyinosine, 2-deoxyinosine being preferred). Unless the context dictates otherwise, the term “primer” also refers to a mixture of primer molecules that is created when mixed bases are included in the primer design and construction to allow them to hybridize to variant sequences in the target nucleic acid molecules. The target sequence variants can be inter- or intra-species variants. Standard nomenclature for mixed bases is shown in Table 1:

TABLE 1
Mixed Base Nomenclature

| | |
|---|---|
| R | A, G |
| Y | C, T |
| M | A, C |
| K | G, T |
| S | C, G |
| W | A, T |
| H | A, C, T |
| B | C, G, T |
| V | A, C, G |
| D | A, G, T |
| N | A, C, G, T |

Preferably, each primer contains no more than three mixed bases in the region of complementarity to a target nucleic acid. In some embodiments, a primer contains zero, one, two or three mixed bases in the region of complementarity to a target nucleic acid.

Primer Pair: A forward and reverse primer pair (each of which can be a mixture of primers with sequence variations to account for possible variations in the target sequence) that is capable of hybridizing with and initiating a DNA polymerization reaction from different strands of the same nucleic acid molecule within a region of less than 5,000 base pairs. In certain embodiments, the primer pair is capable of hybridizing with an initiating a DNA polymerization reaction from different strands of the same nucleic acid molecule within a region of less than 2,500 base pairs or less than 1,500 base pairs.

Sample: The term “sample” as used herein refers to any sample containing or suspected of containing a nucleic acid of interest, for example a genome, a genome fragment, an amplicon corresponding to a region of a genome, or another target nucleic acid. A sample can be subjected to one or more processes and still be considered a “sample.” For example, a sample that is subjected to a PCR amplification reaction remains a “sample” after the PCR amplification reaction.

Single amplicon: The term “single amplicon” as used herein refers to a nucleic acid molecule or group of nucleic acid molecules produced by a PCR amplification reaction from a single organism with a single primer pair. Typically, a “single amplicon” refers to a PCR product with a unique sequence, but can also refer to a PCR product with a group, e.g., a pair, of unique sequences, for example when the organism is heterozygous for the sequence being amplified.

Specific: The term “specific” as used herein in regards to binding of an probe molecule to an amplicon means that the probe molecule has a greater affinity for its target amplicon than other, non-homologous amplicons, typically with a much great affinity, but does not require that the probe molecule is absolutely specific for its target. Thus, a probe molecule can, for example, be capable of hybridizing to an amplicon comprising a first genomic sequence and an amplicon comprising a second, homologous genomic sequence that differs by one or more nucleotides from the first genomic sequence.

Target Strand 1: Target Strand 1 refers to the strand in a double-stranded target nucleic acid to which an Unextended Primer in an Asymmetric Primer Pair is complementary.

Target Strand 2: Target Strand 2 refers to the strand in a double-stranded target nucleic acid to which the “A” region in an Extended Primer in an Asymmetric Primer Pair is complementary.

Unextended Primer: A PCR primer that consists essentially of a nucleotide sequence having at least 75% sequence identity to a corresponding region in Target Strand 2 or at least 75% sequence complementarity to a corresponding region in Target Strand 1.

The term “consisting essentially of” in reference to the Unextended Primer means that the nucleotide sequence may contain no more than 3 additional nucleotides 5′ to the region of (at least 75%) complementarity to the target sequence.

### 6.2. Methods of Distinguishing Between Homologous Genomic Sequences Using Virtual Probes

The present disclosure provides methods of distinguishing between a first genomic sequence from a first organism and a second, homologous genomic sequence from a second organism. The methods allow the identification of an organism present in a sample using virtual probes. A virtual probe for a genomic sequence generally comprises two or more probe molecules that can be distinguished, e.g., by virtue of their discrete locations on an addressable array, or by differential labeling, e.g., with different fluorescent moieties. For convenience, the readout from an individual probe molecule within a virtual probe is sometimes referred to herein as a “signal.” For clarity, a probe molecule need not be labeled to generate a “signal”. For example, the absence of hybridization to a fluorescently labeled amplicon can constitute a “signal”.

Each virtual probe for a genomic sequence contains at least one probe molecule (of the plurality of probe molecules that make up the virtual probe) that is capable of specifically hybridizing to a target nucleic acid (e.g., an amplicon) corresponding to the genomic sequence. In some instances, two or more probe molecules in the virtual probe are capable of hybridizing to a target nucleic acid (e.g., an amplicon) corresponding to the genomic sequence. The hybridization patterns of the probe molecules in a virtual probe to different target nucleic acids (e.g., amplicons) from related genomic sequences are sufficiently different so as to distinguish target nucleic acids from the related genomic sequences, for example distinguish between a first amplicon set from a first genome and a second amplicon set from a second genome with a homologous genomic sequence. The methods can be used, for example, to determine the presence of specific species or strains of bacteria in a sample from which probed amplicons were amplified, directly (e.g., where the sample is directly utilized in PCR) or indirectly (e.g., through an intermediate purification or enrichment step, such as a bead beating method described in Section 6.2.1). Various embodiments disclosed herein describe probing the product of a DNA amplification reaction such as a PCR reaction with a virtual probe; however, it should be understood that probing can alternatively be performed using a method capable of detecting non-amplified genomic DNA.

Exemplary methods for detecting non-amplified genomic DNA are described in Detection of Non-Amplified Genomic DNA, 2012, Spoto and Corradini (eds) doi.org/10.1007/978-94-007-1226-3, the contents of which are incorporated by reference in their entirety. Such methods include optical detection methods (see, e.g., Li and Fan, 2012, “Optical Detection of Non-amplified Genomic DNA,” pp. 153-183 in Detection of Non-Amplified Genomic DNA), electrochemical detection methods (see, e.g., Marin and Merkoçi, 2012, “Electrochemical Detection of DNA Using Nanomaterials Based Sensors,” pp. 185-201 in Detection of Non-Amplified Genomic DNA), piezoelectric sensing methods (see, e.g., Minunni, 2012, “Piezoelectric Sensing for Sensitive Detection of DNA,” pp. 203-233 in Detection of Non-Amplified Genomic DNA), surface plasmon resonance-based methods (see, e.g., D'Agata and Spoto, 2012, “Surface Plasmon Resonance-Based Methods,” pp. 235-261 in Detection of Non-Amplified Genomic DNA), and parallel optical and electrochemical methods (see, e.g., Knoll et al., 2012, “Parallel Optical and Electrochemical DNA Detection,” pp. 263-278 in Detection of Non-Amplified Genomic DNA). Thus, in some embodiments, probing of a sample is performed in the absence of a DNA amplification step (e.g., where the sample contains or is suspected of containing a target nucleic acid which is a genome fragment).

Methods of determining the presence of a first genome from a first organism or a second genome from a second organism, if either is present in a sample, can comprise a step of performing a PCR amplification reaction (e.g., as described in Section 6.2.3) on the sample using PCR primers capable of hybridizing to, and initiating a PCR amplification from, the first genome and the second genome. Amplification from the first genome, if present in the sample, results in a first amplicon set. Amplification from the second genome, if present in the sample, results in a second amplicon set. The PCR amplification products can be probed with a virtual probe to determine the presence or absence of the first amplicon set and second amplicon set. The probing can be performed during the PCR amplification reaction (e.g., when using real-time PCR, for example as described in Section 6.2.3.5) or after the PCR amplification reaction (e.g., by using an array comprising oligonucleotide probe molecules, for example as described in Section 6.3). When the probing is performed after the PCR reaction, for example on an array, it is useful to include fluorescently labeled nucleotides in the PCR mixture to label the resulting PCR amplicons. The location(s) of the fluorescent label on the addressable array and in some instances its intensity can constitute the signals for the probe molecules that make up the virtual probe.

If the first amplicon set is determined to be present, it can be concluded that the sample contains the first genome. Likewise, if the second amplicon set is determined to be present, it can be concluded that the sample contains the second genome. Virtual probes can be used to distinguish between a first amplicon set and a second amplicon set prepared from related microorganisms, for example coagulase negative and coagulase positive Staphylococcus species (e.g., as described in Section 6.2.5.1), S. gordonii and S. anginosus (e.g., as described in Section 6.2.5.2), or S. mitis and S. pneumoniae (e.g., as described in 6.2.5.3).

Samples can be, for example, biological samples, environmental samples, or food products. In some embodiments, the samples are infected with, or at risk of infection with one or more microorganisms. Exemplary samples are described in Section 6.2.1.

The use of methods of the disclosure to distinguish between any homologous genomic sequences (and amplicons corresponding to the homologous genomic sequences) is contemplated. When determining if a species of bacteria or a related species of bacteria is likely to be present in a sample, a virtual probe capable of distinguishing between target nucleic acids (e.g., amplicons) corresponding to genomic sequences encoding rRNA (e.g., 16S rRNA or 23S rRNA), or intergenic spacer regions between rRNA genes (e.g. a 16S rRNA-23S rRNA intergenic spacer region) can be used. Features of exemplary homologous genomic sequences that can be distinguished by the methods of the disclosure are described in Section 6.2.2.

Amplicons for probing with virtual probes according to the methods of the disclosure can be produced by performing a PCR amplification reaction on a sample containing or suspected or at risk of containing a first organism and/or second organism using PCR primers capable of hybridizing to, and initiating a PCR amplification from, the genome of the first organism and the genome of the second organism. The PCR amplification reaction can be performed with a single set of primers (which should produce a first amplicon and second amplicon, respectively, when the first and second organisms are present in the sample). Alternatively, the PCR amplification reaction can be performed with more than one set of primers to produce multiple amplicons corresponding to the first genome and multiple amplicons corresponding to the second genome, when the first and second organisms, respectively, are present in the sample. Exemplary PCR amplification reactions that can be used in the methods of the disclosure are described in Section 6.2.3. Nucleic acid amplification techniques other than PCR (e.g., isothermal amplification techniques), such as loop mediated isothermal amplification (LAMP), nucleic acid sequence based amplification (NASBA), strand displacement amplification (SDA), and rolling circle amplification (RCA), can also be used to prepare amplicons (see, e.g., Fakruddin et al., 2013, J Pharm Bioallied Sci. 5(4): 245-252. Thus, it should be understood that embodiments described herein as being applicable to PCR amplification products are likewise applicable to amplification products produced using an alternative amplification method.

Exemplary features of probe molecules that can be used in virtual probes, and exemplary features of virtual probes are described in Sections 6.2.4 and 6.2.5, respectively.

In some embodiments, the probing of PCR amplification products comprises the steps of contacting the PCR amplification products with an array, e.g., as described in Section 6.3, washing unbound nucleic acid molecules from the array, and measuring the signal intensity of a label (e.g., a fluorescent label) at each probe molecule location on the array.

In other embodiments, the probing of the PCR amplification products comprises measuring signals from oligonucleotide probe molecules used in a real-time PCR reaction.

Systems that can be used to perform the methods of the disclosure are described in Section 6.4.

Kits that can be used in the methods of the disclosure are described in Section 6.5.

6.2.1. Samples

The sample used in the methods of the disclosure can be any type or form of sample that contains genomic DNA that is in a condition, or can be prepared to be in a condition, suitable for PCR amplification. In certain embodiments, the sample is at risk of infection with one or more microorganisms, for example, one or more species of microorganisms. In other embodiments, the sample is suspected of having an infection with one or more microorganisms, for example, one or more species of microorganisms. The sample can be, for example, a biological sample, an environmental sample, or a food product.

Examples of samples include various fluid samples. In some instances, the sample can be a bodily fluid sample from a subject. The sample can include tissue collected from a subject. The sample can include a bodily fluid, secretion, and/or tissue of a subject. The sample can be a biological sample. The biological sample can be a bodily fluid, a secretion, and/or a tissue sample. Examples of biological samples include but are not limited to, blood, serum, saliva, urine, gastric and digestive fluid, tears, stool, semen, vaginal fluid, interstitial fluids derived from tumorous tissue, ocular fluids, sweat, mucus, earwax, oil, glandular secretions, breath, spinal fluid, hair, fingernails, skin cells, plasma, nasal swab or nasopharyngeal wash, spinal fluid, cerebrospinal fluid, tissue, throat swab, wound swab, biopsy, placental fluid, amniotic fluid, cord blood, emphatic fluids, cavity fluids, sputum, pus or other wound exudate, infected tissue sampled by wound debridement or excision, cerebrospinal fluid, lavage, leucopoiesis specimens, peritoneal dialysis fluid, milk and/or other excretions.

A subject can provide a sample, and/or a sample can be collected from a subject. The subject can be a human or a non-human animal. The sample can be collected from a living or dead subject. The animal can be a mammal, such as a farm animal (e.g., cow, pig, sheep), a sport animal (e.g., horse), or a pet (e.g., dog or cat). The subject can be a patient, clinical subject, or pre-clinical subject. A subject can be undergoing diagnosis, treatment, and/or disease management or lifestyle or preventative care. The subject may or may not be under the care of a health care professional.

In some embodiments, the sample can be an environmental sample. Examples of environmental samples include air samples, water samples (e.g., groundwater, surface water, or wastewater), soil samples, and plant samples.

Additional samples include food products, beverages, manufacturing materials, textiles, chemicals, and therapies.

In some embodiments, the sample is a sample containing or suspected of containing a pathogen such as, for example, one or more of Mycobacterium tuberculosis, Mycobacterium avium subsp paratuberculosis, Staphylococcus aureus (including methicillin sensitive and methicillin resistant Staphylococcus aureus (MRSA)), Staphylococcus epidermidis, Staphylococcus lugdunensis, Staphylococcus maltophilia, Streptococcus pyogenes, Streptococcus pneumoniae, Streptococcus agalactiae, Haemophilus infuenzae, Haemophilus parainfuluezae, Moraxella catarrhalis, Klebsiella pneumoniae, Klebsiella oxytoca, Escherichia coli, Pseudomonas aeruginosa, Acinetobacter sp., Bordetella pertussis, Neisseria meningitidis, Bacillus anthracis, Nocardia sp., Actinomyces sp., Mycoplasma pneumoniae, Chlamydia pneumonia, Legionella species, Pneumocystis jiroveci, influenza A virus, cytomegalovirus, rhinovirus, Enterococcus faecium, Acinetobacter baumannii, Corynebacterium amycolatum, Enterobacter aerogenes, Enterococcus faecalis Cl 4413, Enterobacter cloacae, Serratia marcescens, Streptococcus equi, Candida albicans, Proteus mirabilis, Micrococcus luteus, Stenotrophomonas (Xanthomonas) maltophilia, and Salmonella sp. In some embodiments, the sample is a sample containing or suspected of containing an Enterobacteriaceae group bacteria such as Enterobacter aerogenes, Enterobacter asburiae, or Enterobacter hormaechei.

A sample can be pre-processed prior to performing PCR amplification. Thus, the sample subjected to PCR amplification in the methods of the disclosure can be a sample which is, for example, processed, extracted, or fractionated from any of the types of samples described in this Section or elsewhere in the disclosure (e.g., a sample processed, extracted or fractionated from urine, sputum, a wound swab, blood, or peritoneal dialysis fluid).

Examples of pre-processing steps that can be used include filtration, distillation, extraction, concentration, centrifugation, inactivation of interfering components, addition of reagents, and the like, as discussed herein or otherwise as is known in the art.

It can be particularly advantageous to remove unwanted cell types and particulate matter from biological samples to maximize recovery of genomic DNA from a cell type of interest prior to PCR.

If the intent is to detect bacteria in a biological sample, then it can be desirable to pre-process the biological sample through a filter so that particulates and non-bacterial cells are retained on a filter while bacterial cells (including their spores, if desired) pass through. A “filter,” as used herein, is a membrane or device that allows differential passage of particles and molecules based on size. Typically this is accomplished by having pores in the filter of a particular nominal size. For instance, filters of particular interest for bacterial detection applications have pores sufficiently large to allow passage of bacteria but small enough to prevent passage of eukaryotic cells that present in a sample of interest. Generally, bacterial cells range from 0.2 to 2 μm (micrometers or microns) in diameter, most fungal cells range from 1 to 10 μm in diameter, platelets are approximately 3 μm diameter and most nucleated mammalian cells are typically 10 to 200 μm in diameter. Therefore, filter pore sizes of less than 2 μm or less than 1 μm are particularly suitable for removing non-bacterial cells from a biological sample if detection of bacteria is intended.

In addition to or in lieu of a filtration step, a biological sample can be subject to centrifugation to remove cells and debris from a sample. Centrifugation parameters that precipitate eukaryotic but not bacterial cells are known in the art. The supernatant can then be filtered if desired.

Samples can be prepared for PCR amplification using any of the various processes for preparing samples comprising genomic DNA for PCR which are known in the art (e.g., following one or more of the pre-processing steps described above). In some embodiments, a commercially available DNA extraction reagent, kit, and/or instrument can be used, e.g., a QIAamp DNA Mini Kit (Qiagen), a MagMAX™ DNA Multi-Sample Kit (ThermoFisher Scientific), a Maxwell® RSC Instrument (Promega), etc.

In some embodiments, a sample is prepared for PCR by a process comprising bead-beating, for example as described in U.S. Pat. No. 10,036,054, the contents of which are incorporated herein by reference in their entireties. Blood can be directly subjected to bead beating after being collected in a commercially available blood collection tube, for example by adding bead beating beads to the collection tube and subjecting the collection tube to agitation. Examples of commercially available collection tubes that can be used to collect blood samples include lavender-top tubes containing EDTA, light blue-top tubes containing sodium citrate, gray-top tubes containing potassium oxalate, or green-top tubes containing heparin.

6.2.2. Homologous Genomic Sequences

The methods of the disclosure can be used to identify and/or distinguish first and second homologous genomic sequences (and target nucleic acids such as amplicons corresponding to the first and second homologous genomic sequences). Homologous genomic sequences are genomic sequences found in species or strains which have shared ancestry but which are not identical in nucleotide sequence. Thus, for example, homologous genomic sequences are found in closely related species or strains of bacteria.

The first genomic sequence and the second genomic sequence are generally genomic sequences from a first microorganism and a second microorganism (e.g., bacteria, viruses, or fungi). The first and/or second microorganisms can be, for example, a human pathogen and/or an animal pathogen. The microorganisms can be from the same order, the same family, the same genus, the same group, or even the same species. In preferred embodiments, the first and second microorganism are bacteria.

Advances in sequencing technologies have led to a substantial increase in the number of whole bacterial genomic sequences available in a number of public database repositories, such as the National Center for Biotechnology Information (NCBI), European Molecular Biology Laboratory (EMBL) and the DNA Databank of Japan (DDBJ), and such databases can be used to identify homologous genomic sequences.

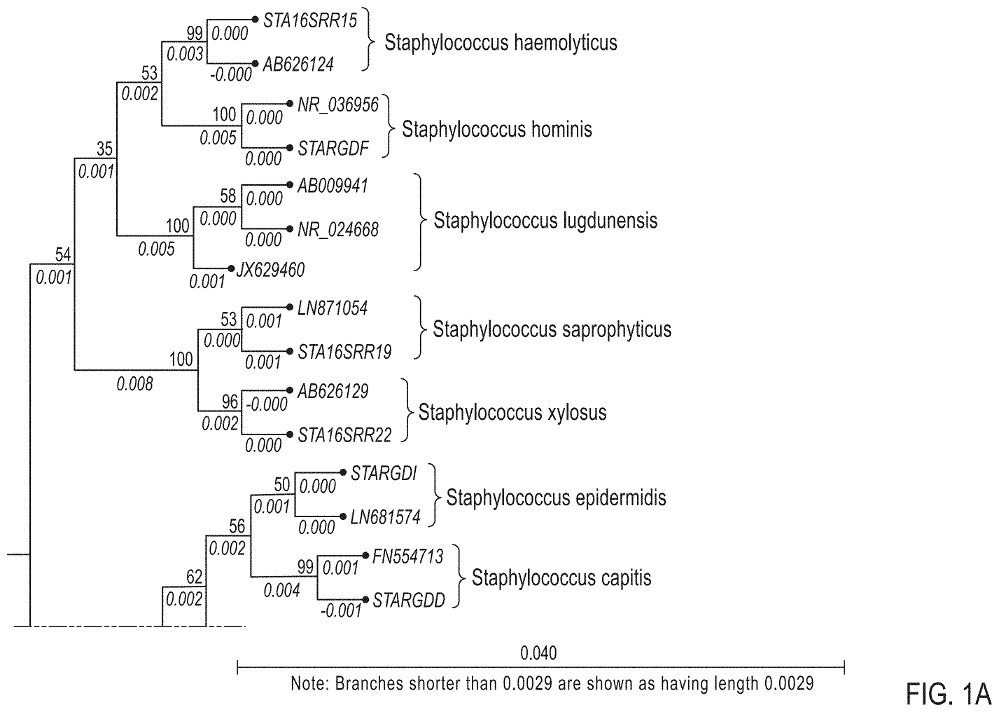

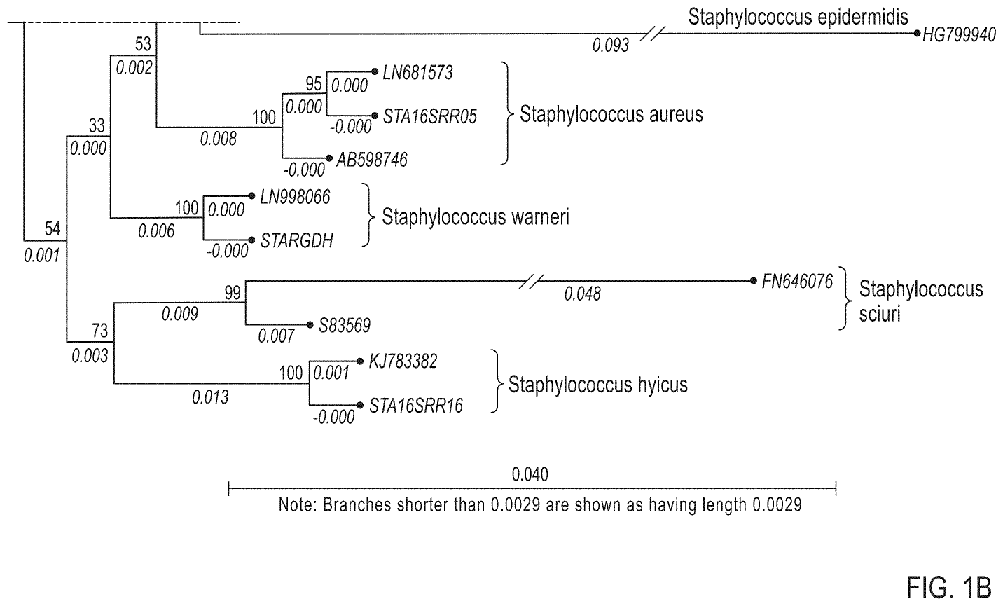

Homologous genomic sequences in closely related microorganisms are often found in genes encoding rRNA and intergenic spacer regions between genes encoding rRNA. Sequence comparisons of bacterial species have long been carried out using the genes for the 16S ribosomal RNA (16S rRNA). The 16S ribosomal RNA genes code for the 16S RNA component of the 30S small subunit of the bacterial ribosome, a protein/RNA complex that is responsible for protein production. The genes comprise regions of highly conserved sequence interspersed with nine hypervariable regions (V1-V9). The sequence variations in the hypervariable regions allow for most of the observable differences between closely related species. Due to the slow rate of sequence evolution observed among these genes, 16S rRNA sequences have been used in constructing phylogenic trees for a number of bacterial species. An exemplary phylogenic tree prepared from the 16S rRNA genes of several Staphylococcus species obtained from GenBank is shown in FIG. 1.

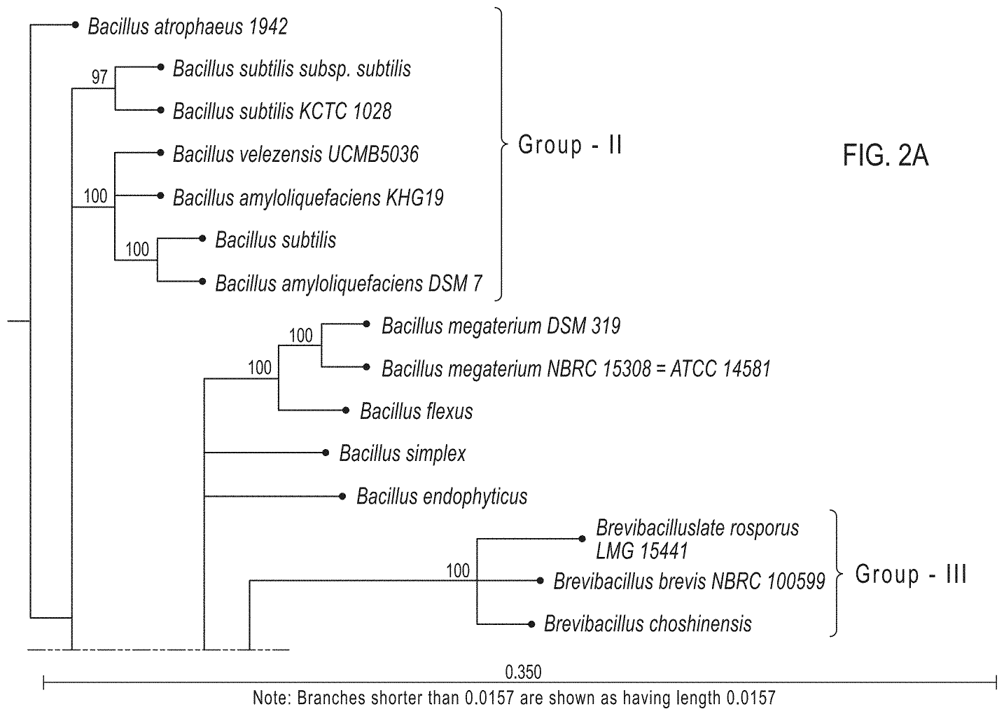

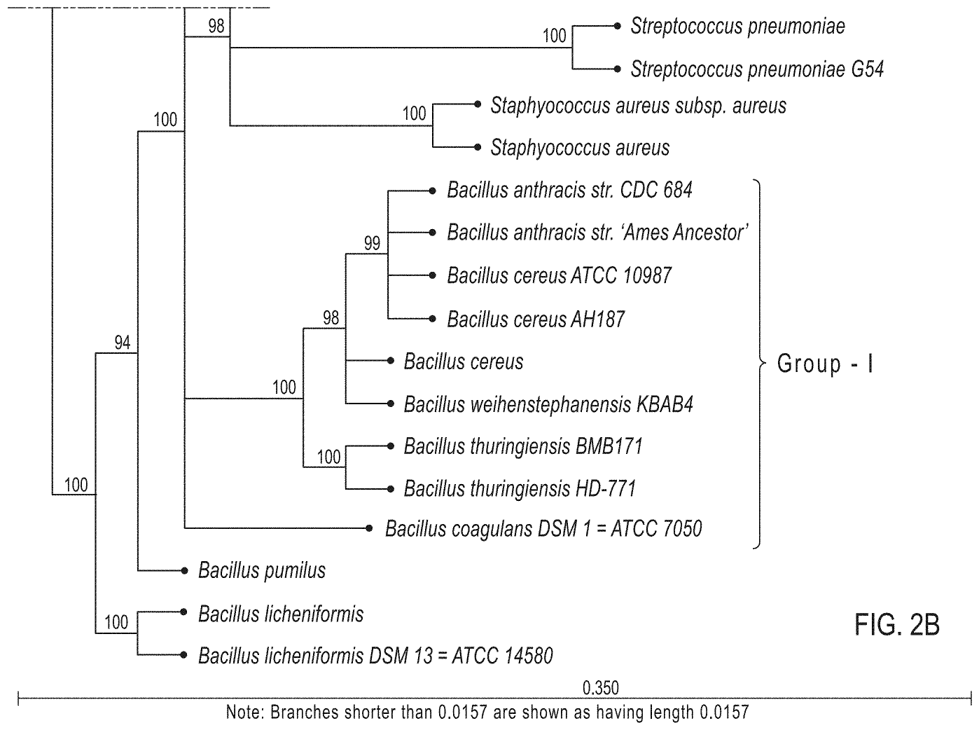

The bacterial genome contains a second ribosomal rRNA gene, the 23S rRNA gene. The 16S rRNA and the 23S rRNA genes are separated from each other by a spacer region known as the 16S-23S internal transcribed spacer region (ITS) or the 16S-23S intergenic spacer region. The 16S-23S rRNA ITS region comprises hypervariable regions comprising species and inter-species specific sequences that can be used for distinguishing and identifying particular bacterial species (K. Okamura, et al., 2012). An exemplary phylogenic tree for the Streptococcus viridians group created from multiple alignment of 16s rRNA and 16s-23s rRNA genome sequences is shown in FIG. 2.

In some embodiments of the methods of the disclosure, the first genomic sequence and the second genomic sequence each comprises a nucleotide sequence of a gene encoding rRNA. In other embodiments, the first genomic sequence and the second genomic sequence each comprise a nucleotide sequence of an intergenic spacer region between rRNA genes.

In embodiments in which the microorganisms are bacteria, the first genomic sequence and the second genomic sequence can each comprise, for example, a nucleotide sequence of a 16S rRNA gene or a 23S rRNA gene. In some embodiments, the first genomic sequence and the second genomic sequence each comprises a nucleotide sequence of a 16S rRNA gene. In other embodiments, the first genomic sequence and the second genomic sequence each comprises a nucleotide sequence of a 23S rRNA gene. In other embodiments, the genomic sequence comprises a nucleotide sequence found in a 16S-23S intergenic spacer region.

In certain specific embodiments, the first genomic sequence and/or the second homologous genomic sequence are genomic sequences from pathogens, e.g., bacteria, viruses or fungi, that can be found in human blood, urine or peritoneal fluid. Examples of such pathogens include, but are not limited to, Mycobacterium tuberculosis, Mycobacterium avium subsp paratuberculosis, Staphylococcus aureus (including methicillin sensitive and methicillin resistant Staphylococcus aureus (MRSA)), Staphylococcus epidermidis, Staphylococcus lugdunensis, Staphylococcus maltophilia, Streptococcus pyogenes, Streptococcus pneumoniae, Streptococcus agalactiae, Haemophilus influenzae, Haemophilus parainfuluezae, Moraxella catarrhalis, Klebsiella pneumoniae, Klebsiella oxytoca, Escherichia coli, Pseudomonas aeruginosa, Acinetobacter sp., Bordetella pertussis, Neisseria meningitidis, Bacillus anthracis, Nocardia sp., Actinomyces sp., Mycoplasma pneumoniae, Chlamydia pneumonia, Legionella species, Pneumocystis jiroveci, influenza A virus, cytomegalovirus, rhinovirus, Enterococcus faecium, Acinetobacter baumannii, Corynebacterium amycolatum, Enterobacter aerogenes, Enterococcus faecalis Cl 4413, Enterobacter cloacae, Serratia marcescens, Streptococcus equi, Candida albicans, Proteus mirabilis, Micrococcus luteus, Stenotrophomonas (Xanthomonas) maltophilia, and Salmonella sp.

### 6.2.3. PCR Amplification

In some embodiments of the methods of the disclosure, PCR amplification is performed on a sample using PCR primers capable of hybridizing to, and initiating a PCR amplification from, a genome that may be present in the sample. The PCR amplification reaction can be a “symmetric” PCR reaction, that is, the reaction makes double-stranded copies of template DNA by utilizing a forward primer and a reverse primer designed to have “melting temperatures,” or “Tₘ's” that equal or within a few ° C. of one another. Commonly used computer software programs for primer design warns users to avoid high Tₘ difference, and have automatic Tₘ matching features. “Asymmetric” PCR reactions that can make single-stranded DNA amplicons can also be used. Real-time PCR reactions can also be used. In the context of a PCR amplification reaction, a genomic sequence amplified by the reaction can be referred to as a “target” nucleic acid, a “template” nucleic acid, or the like.

PCR amplification reactions can use a single primer set or multiple primer sets (e.g., when the PCR amplification is a multiplex PCR). Multiplex PCR can be useful, for example, to produce an amplicon corresponding to a first genomic sequence (e.g., a 16S rRNA gene) and/or a different amplicon corresponding to a second genomic sequence (e.g., a 23S rRNA gene). As an alternative to multiplex PCR, amplicons produced by separate PCR amplification reactions performed with different primer sets can be pooled for subsequent analysis. Advantageously, a single primer set can be used to make amplicons corresponding to homologous genomic sequences found in the genome of multiple strains or species, e.g., 2, 3, 4, or more than 4 species, which can be, for example, members of the same genus.

PCR amplification conditions can be selected (whether symmetric or asymmetric, singleplex or multiplex), for example, such that the DNA amplification products are 100 to 1000 nucleotides in length. In some embodiments, the PCR reactions are selected so that the DNA amplification products are 300 to 800 nucleotides in length. In other embodiments, the PCR conditions are selected so that the DNA amplification products are 400 to 600 nucleotides in length.

In some embodiments, PCR amplification reactions used in the methods of the disclosure incorporate a label that produces a measurable signal into any amplicons produced by the reaction. The label can be, for example, a fluorescent label, an electrochemical label, or a chemiluminescent label. Fluorescent labeling can be achieved by fluorescently labeled nucleotide incorporation during PCR and/or by the use of labeled primers for PCR. Electrochemical labeling can be achieved by redox-active labeled nucleotide incorporation during PCR and/or by the use of redeox-active labeled primers for PCR (see, e.g., Hocek and Fojta, 2011, Chem. Soc. Rev., 40:5802-5814; Fojta, 2016, Redox Labeling of Nucleic Acids for Electrochemical Analysis of Nucleotide Sequences and DNA Damage. In: Nikolelis D., Nikoleli GP. (eds) Biosensors for Security and Bioterrorism Applications. Advanced Sciences and Technologies for Security Applications. Springer, Cham). Chemiluminescent labeling can be achieved, for example, by the use of biotin labeled primers for PCR, binding a streptavidin-alkaline phosphatase conjugate, then incubating with a chemiluminescent 1,2-dioxetane substrate.

Examples of suitable fluorescent moieties include FITC, EDANS, Texas red, 6-joe, TMR, Alexa 488, Alexa 532, BODIPY FL/C3, BODIPY R6G, BODIPY FL, Alexa 532, BODIPY FL/C6, BODIPY TMR, 5-FAM, BODIPY 493/503, BODIPY 564, BODIPY 581, Cy3, Cy5, R110, TAMRA, Texas red, and x-Rhodamine.

Fluorescent moieties can be attached to dNTPs, particularly those containing cytosine as a base (cytidylic acid, cytidine 5′-phosphate, cytidine 5′-diphosphate, cytidine 5′-triphosphate, or a polymer thereof, or a polymer containing cytidylic acid).

The position of the dNTP labeling can be at the base (amino group), phosphate group (OH group), or deoxyribose moiety (2′- or 3′-OH group). The preferred position is at the base.

Like other nucleotides, fluorescently labeled dNTPs can be incorporated into both strands of a PCR amplicon at random sites, typically dC sites, and extended by DNA polymerase.

Fluorescent dNTPs are commercially available in highly concentrated form and can be added to the PCR reaction mixture without adjusting the concentration of each unlabeled dNTP. For most PCR amplifications, the typical ratio of dNTP to fluorescent dNTPs is between 100:1 and 1000:1. Thus, to fluorescently labeled dNTPs can be included among the PCR Reagents at 0.1% to 1% the (molar) quantity of the unlabeled dNTPs.

Detection of fluorescently labeled PCR products can be achieved through hybridization to probe molecules, for example probe molecules bound to a microarray. A suitable microarray system takes advantage of three-dimensional crosslinked polymer networks, as described in U.S. Pat. No. 9,738,926, the contents of which are incorporated by reference herein in their entireties

6.2.3.1. Primers

The primers utilized in a PCR reaction are designed to recognize and hybridize to the sequence of a given nucleic acid template(s), e.g., a target genomic sequence(s). Mismatches in the sequence of a primer and a target nucleic acid template may result in reduced efficiency of the PCR reaction and/or amplification of a sequence other than the desired sequence. Parameters for successful primer design are well known in the art (see for example, Dieffenbach, et al., 1993) and include primer length, melting temperature, GC content, and the like. PCR primers do not need to share 100% sequence identity with a given target nucleic acid template, and PCR primers having at least 75%, e.g., 80%, e.g., 85%, e.g., 90%, e.g., 95%, e.g., 96%, e.g., 97%, e.g., 98%, e.g., 99%, or 99.5% identity with a target sequence may function to hybridize to and allow amplification of a target sequence.

The present disclosure provides for additional parameters suitable for preparing a unique primer system with high specificity and good amplification efficiency. Primers are typically 18 to 24 bases in length, but can be longer, e.g., 25 to 50 bases in length, e.g., 25 to 45 bases in length, e.g., 30 to 45 bases in length, e.g., 35 to 45 bases in length, e.g., 40 to 45 bases in length, or e.g., 40 to 50 bases in length. Primers used in PCR amplification are often designed in pairs, with one primer referred to as the “forward” primer and one primer referred to as the “reverse” primer. Forward primers of the present disclosure can be designed with G and/or C residues at the 3′ end so as to provide a “GC-clamp”. G and C nucleotide pairs exhibit stronger hydrogen bonding than A-T nucleotide pairs; as such, a GC-clamp at the 3′ end of a primer may aid in increasing sequence specificity, increasing the likelihood of hybridization, and increasing the overall efficiency of the PCR reaction.

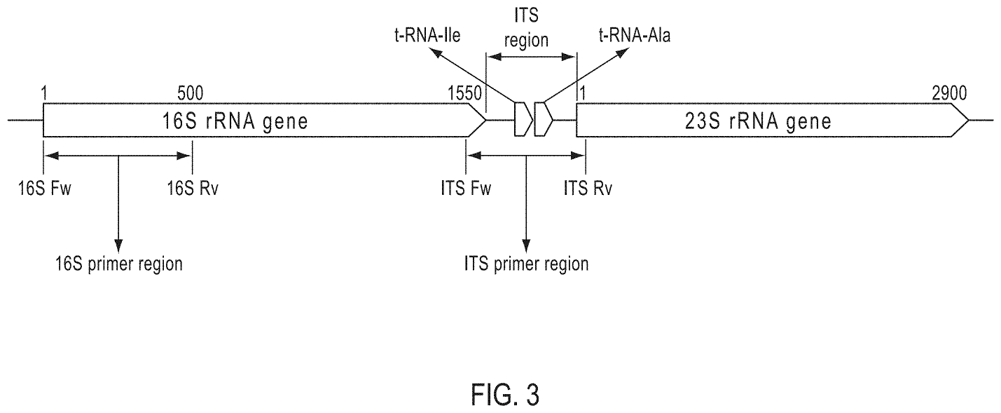

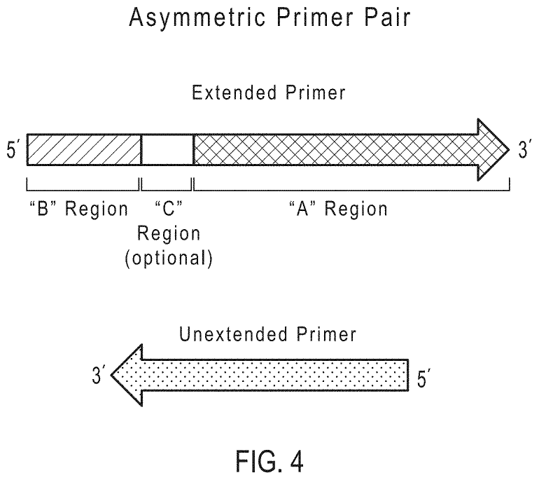

A set of primers can be designed to amplify two genomic regions, for example, a set of primers can include one primer pair specific to the 16S rRNA gene and a second primer pair specific to the 16S-23S rRNA ITS region (see FIG. 3). Such primer sets can be used, for example, to produce multiple amplicons in a single PCR reaction.

PCR primer pairs can be designed to amplify a sequence conserved across a number of species, for example, to amplify the 16S rRNA genes of multiple bacterial species. Thus, it can be possible to produce amplicons corresponding to homologous genomic sequences using a single PCR primer pair, which is advantageous when performing PCR on a sample containing or suspected of containing one of a number of possible organisms. Parameters for primers designed to amplify a conserved sequence can include identifying a conserved region across the various species, optionally verifying as correct any sequence differences in the conserved region (e.g., if there is uncertainty whether a published sequence is correct), and selecting a sequence that is at least 75%, e.g., 80%, e.g., 85%, e.g., 90%, e.g., 95%, e.g., 96%, e.g., 97%, e.g., 98%, e.g., 99%, or even 100% conserved across the sequences. Primers exhibiting less than 100% sequence identity may simply contain one or more single nucleotide bases that are different from a given template, that is, all primers in the preparation contain the same sequence to each other. Alternately, primers can be prepared to contain alternate nucleotide residues at a particular location in the sequence. For example, a reverse primer for the amplification of the 16S region of several species can comprise a pool of oligonucleotides, a percentage of which, e.g., 50%, contain a first nucleotide at a position in the primer and a percentage of which, e.g., 50%, contain a second nucleotide at the position.

In some embodiments, the primers utilized in the methods of the disclosure are labeled with a detectable label (e.g., a fluorescent label). For example, in some embodiments at least one primer is 5′ fluorescently labeled. In other embodiments more than one primer is 5′ fluorescently labeled. Fluorescent labels suitable for labeling primers are known in the art, and include Cy5, FAM, JOE, ROX and TAMRA.

### 6.2.3.2. Symmetric PCR Amplification

A typical three-step PCR protocol that can be used in the methods of the disclosure (see PCR PROTOCOLS, a Guide to Methods and Applications, Innis et al. eds., Academic Press (San Diego, Calif. (USA) 1990, Chapter 1) may include denaturation, or strand melting, at 93-95° C. for more than 5 sec, primer annealing at 55-65° C. for 10-60 sec, and primer extension for 15-120 sec at a temperature at which the polymerase is highly active, for example, 72° C. for Taq DNA polymerase. A typical two-step PCR protocol may differ by having the same temperature for primer annealing as for primer extension, for example, 60° C. or 72° C. For either three-step PCR or two-step PCR, amplification involves cycling the reaction mixture through the foregoing series of steps numerous times, typically 25-40 times. During the course of the reaction the times and temperatures of individual steps in the reaction may remain unchanged from cycle to cycle, or they may be changed at one or more points in the course of the reaction to promote efficiency or enhance selectivity.

In addition to the pair of primers and target nucleic acid a PCR reaction mixture typically contains each of the four deoxyribonucleotide 5 triphosphates (dNTPs), typically at equimolar concentrations, a thermostable polymerase, a divalent cation (typically Mg²⁺), and a buffering agent. The volume of such reactions is typically 20-100 μl. Multiple target sequences can be amplified in the same reaction. The number of cycles for a particular PCR amplification depends on several factors including: a) the amount of the starting material, b) the efficiency of the reaction, and c) the method and sensitivity of detection or subsequent analysis of the product. Cycling conditions, reagent concentrations, primer design, and appropriate apparatuses for typical cyclic amplification reactions are well known in the art (see, for example, Ausubel, F. Current Protocols in Molecular Biology (1988) Chapter 15: “The Polymerase Chain Reaction,” J. Wiley (New York, N.Y. (USA)).

### 6.2.3.3. Asymmetric PCR Amplification

Exemplary asymmetric PCR methods are described in Gyllensten and Erlich, 1988, Proc. Natl. Acad. Sci. (USA) 85: 7652-7656 (1988); and Gyllensten and Erlich, 1991, U.S. Pat. No. 5,066,584. Traditional asymmetric PCR differs from symmetric PCR in that one of the primers is added in limiting amount, typically 1/100^{th} to ⅕^{th} of the concentration of the other primer. Double-stranded amplicon accumulates during the early temperature cycles, as in symmetric PCR, but one primer is depleted, typically after 15-25 PCR cycles, depending on the number of starting templates. Linear amplification of one strand takes place during subsequent cycles utilizing the undepleted primer. Primers used in asymmetric PCR reactions reported in the literature are often the same primers known for use in symmetric PCR. Poddar (Poddar, 2000, Mol. Cell Probes 14: 25-32) compared symmetric and asymmetric PCR for amplifying an adenovirus substrate by an end-point assay that included 40 thermal cycles. He reported that a primers ratio of 50:1 was optimal and that asymmetric PCR assays had better sensitivity that, however, dropped significantly for dilute substrate solutions that presumably contained lower numbers of target molecules.

### 6.2.3.4. Improved Asymmetric PCR Amplification

Improved asymmetric PCR methods are described in U.S. Pat. No. 10,513,730, the contents of which are incorporated herein by reference in their entireties. The improved asymmetric PCR methods include both an exponential phase and a linear phase. During the exponential phase, both strands of the target nucleic acid are amplified. During the linear phase, only one of the strands is amplified, resulting in an excess of a single strand of target nucleic acid.

The improved asymmetric PCR methods achieve the excess of a single strand though the use of primer pairs of different lengths and melting temperatures, with the longer primer referred to as the “Extended Primer” and the shorter primer referred to as the “Unextended Primer”. The Extended Primer has a higher melting temperature than the Unextended Primer and can be used to selectively amplify a single strand of the target nucleic acid using PCR cycles in which the annealing step is performed at a temperature greater than the melting temperature of the Unextended Primer but lower than the melting temperature of the Extended Primer. The selective amplification gives rise to a PCR product mixture that is enriched in the target strand which can be probed in a subsequent detection assay.

The Extended Primers contain in addition to the sequence complementary to the target nucleic acid a 5′ extension containing a sequence that is complementary to the target-binding portion of the same primer. Without being bound by theory, it is believed that the use of the 5′ extension allows intra- or inter-molecular hybridization of Extended Primer molecules and prevents arbitrary or non-specific binding of these longer primers to DNA molecules present in the PCR reaction at the beginning of the PCR reaction. This in turns prevents non-specific DNA amplification and prevents “noise” in the PCR product, which can be problematic when amplifying a target that is present in low quantities in a biological sample.

The initial PCR reaction mixture includes
Nucleic acid sample;
Asymmetric Primer Pair;
Thermostable DNA polymerase; and
PCR Reagents.

The initial concentration of the Extended Primer and the Unextended Primer in the PCR reaction can each range from 200 nM to 8 μM. The Extended Primer and Unextended Primer can be included in equimolar quantities in the initial PCR reaction, e.g., at concentrations ranging between about 200 nM and 1 μM each, for instance at concentrations of 500 nM each. Alternatively, the Extended Primer and Unextended Primer can be included in non-equimolar quantities in the initial PCR reaction. In certain embodiments, the initial concentration of the Extended Primer is preferably in an excess of the concentration of Unextended Primer, for example about a 2-fold to 30-fold molar excess. Accordingly, in certain aspects, the concentration the Extended Primer ranges between about 1 μM and 8 μM and the concentration of the Unextended Primer ranges between about 50 nM and 200 nM.

The Asymmetric Primer Pair can be designed to amplify nucleic acid from any source, and for diagnostic applications the Asymmetric Primer Pair can be design to amplify DNA from pathogens such as those identified in Section 6.2.1.

The Asymmetric Primer Pair can be designed so as to be capable of amplifying homologous nucleic acid sequences present in many species simultaneously, for example the highly conserved 16S ribosomal sequence in bacteria.

Thermostable DNA polymerase: The thermostable polymerases that can be used in the asymmetric PCR reactions of the disclosure includes, but are not limited to, Vent (Tli/Thermoccus Literalis), Vent exo-, Deep Vent, Deep Vent exo-, Taq (Thermus aquaticus), Hot Start Taq, Hot Start Ex Taq, Hot Start LA Taq, DreamTaq™, TopTaq, RedTaq, Taqurate, NovaTaq™, SuperTaq™, Stoffel Fragment, Discoverase™ dHPLC, 9° Nm, Phusion®, LongAmp Taq, LongAmp Hot Start Taq, OneTaq, Phusion® Hot Start Flex, Crimson Taq, Hemo KlenTaq, KlenTaq, Phire Hot Start II, DyNAzyme I, DyNAzyme II, M-MulV Reverse Transcript, PyroPhage, Tth(Thermos termophilus HB-8), Tfl, Amlitherm™ Bacillus DNA, DisplaceAce™, Pfu (Pyrococcus furiosus), Pfu Turbot, Pfunds, ReproFast, PyroBest™, VeraSeq, Mako, Manta, Pwo (pyrococcus, woesei), ExactRun, KOD (thermococcus kodakkaraensis), Pfx, ReproHot, Sac (Sulfolobus acidocaldarius), Sso (Sulfolobus solfataricus), Tru (Thermus ruber, Pfx50™ (Thermococcus zilligi), AccuPrime™ GC-Rich (Pyrolobus fumarius), Pyrococcus species GB-D, Tfi (Thermus filiformis), Tfi exo-, ThermalAce™, Tac (Thermoplasma acidophilum), (Mth (M. thermoautotrophicum), Pab (Pyrococcus abyssi), Pho (Pyrococcus horikosihi, B103 (Picovirinae Bacteriophage B103), Bst (Bacillus stearothermophilus), Bst Large Fragment, Bst 2.0, Bst 2.0 WarmStart, Bsu, Therminator™, Therminator™ II, Therminator™ II, and Therminator™ T. In a preferred embodiment, the DNA polymerase is a Taq polymerase, such as Taq, Hot Start Taq, Hot Start Ex Taq, Hot Start LA Taq, DreamTaq™, TopTaq, RedTaq, Taqurate, NovaTaq™ or SuperTaq™.

An illustrative set of asymmetric cycles for use in the improved asymmetric methods is shown in Table 2.

TABLE 2

| | | | | No. of |
|---|---|---|---|---|
| Phase | Step | Temperature | Time | Cycles |
| Initial | Initial | 90-100° C., | 0-5 minutes, | 0-1 |
| denaturation | denaturation | preferably | preferably 2 | |
| | | 95° C. | minutes | |
| Exponential | Denaturation | 90-100° C., | 15-25 seconds, | 20-40, |
| phase | | preferably | preferably 20 | preferably |
| | | 95° C. | seconds | 30-37 |
| | Annealing | 58° C. | 12-18 seconds, | (e.g., 35) |
| | | | preferably 15 | |
| | | | seconds | |
| | Extension | 72° C. | 30-50 seconds, | |
| | | | preferably 40 | |
| | | | seconds | |
| Linear | Denaturation | 90-100° C., | 15-25 seconds, | 15-25, |
| phase | | preferably | preferably 20 | preferably |
| | | 95° C. | seconds | 20 |
| | Simultaneous | 72° C. | 40-60 seconds, | |
| | annealing and | | preferably 50 | |
| | extension | | seconds | |
| Extended | Extended | 72° C. | 0-5 minutes, | 0-1 |
| extension | extension | | preferably 2 | |
| | | | minutes | |

The ranges of numbers of cycles shown in Table 2 can beused for any Asymmetric Primer Pair, and the optimal number of cycles will depend on the copy number of the target DNA in the initial PCR mixture: the greater the initial copy number the fewer number of cycles are needed in the exponential phase to produce asufficient quantity of PCR products to serve as templates for the linear phase. The optimization of cycle number is routine for the skilled artisan.

The temperatures shown in Table 2 are particularly useful where the Tₘ of the Extended Primer is greater than 72° C. (e.g., 75-80° C.) and the Tₘ of the Unextended Primer is above 58° C. but below 72° C. (e.g., 60-62° C.) and when the thermostable DNA polymerase is active at 72° C.

The cycle times, particularly the extension times, can be varied according to the melting temperatures of the primers and the length of the PCR product, with longer PCR products calling for longer extension times. Arule of thumb is that the extension step should be at least 60 seconds per 1,000 bases of amplicon. The extension step can be extended in the linear phase to provide additional time for annealing.

6.2.3.4.1. Extended Primer

The “A” region of the Extended Primer has at least 75% sequence identity to a corresponding region in Target Strand 1. In certain embodiments, the “A” region of the primer has at least 80%, at least 85%, at least 90%, or at least 95% identical to the corresponding region in Target Strand 1. In yet other embodiments, the “A” region of the primer has 100% sequence identity to the corresponding region of Target Strand 1.

Stated differently, in various embodiments the “A” region of the Extended Primer has at least 75%, at least 80%, at least 85%, at least 90%, or at least 95% or 100% sequence identity to the complement of the corresponding region in Target Strand 2. Typically, the more 5′ any mismatches are between the primer sequence and the target sequence are positioned, the more likely they are to be tolerated during the PCR reaction. One of skill in the art can readily design primer sequences that have less than 100% sequence identity to the target strand but can still efficiently amplify target DNA.

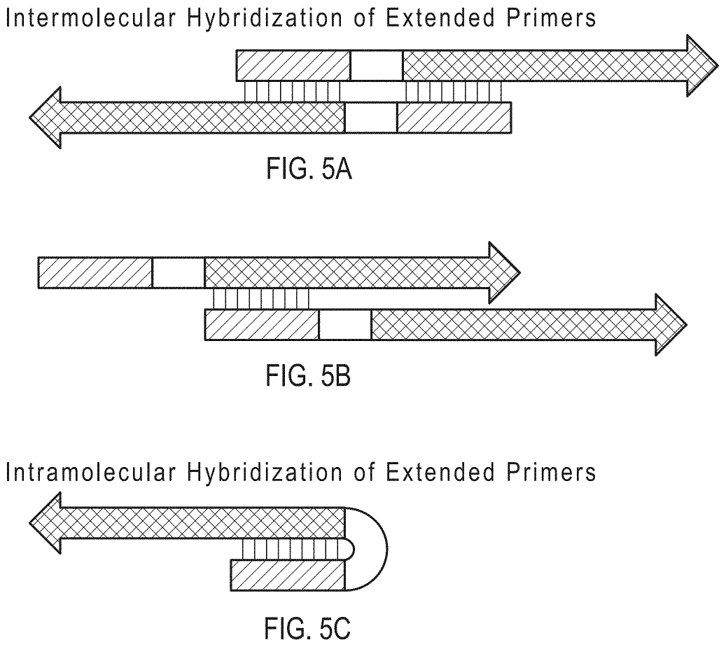

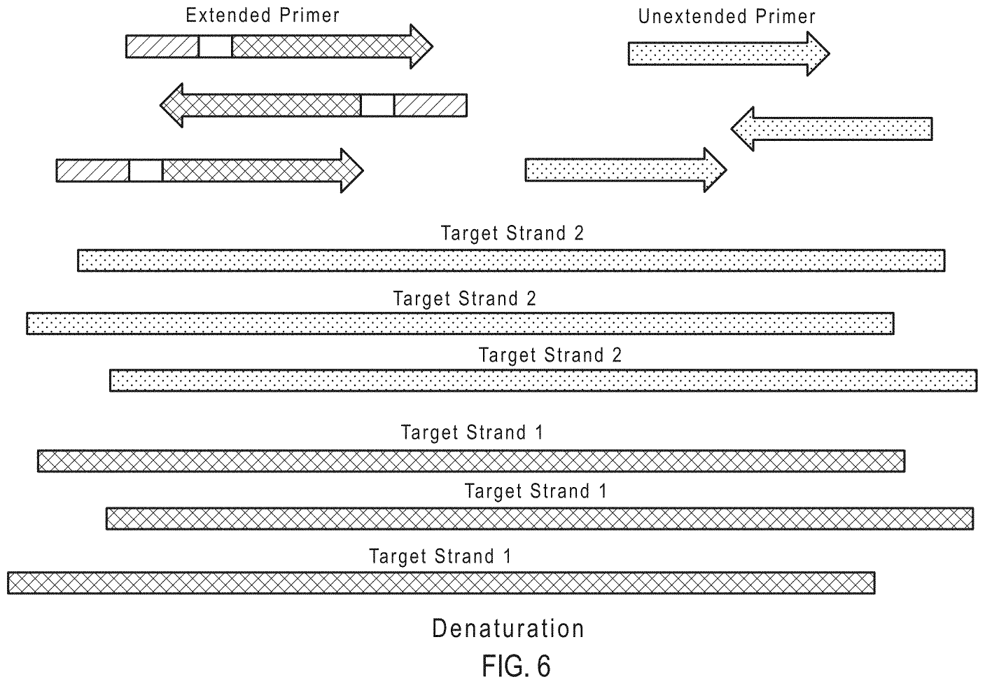

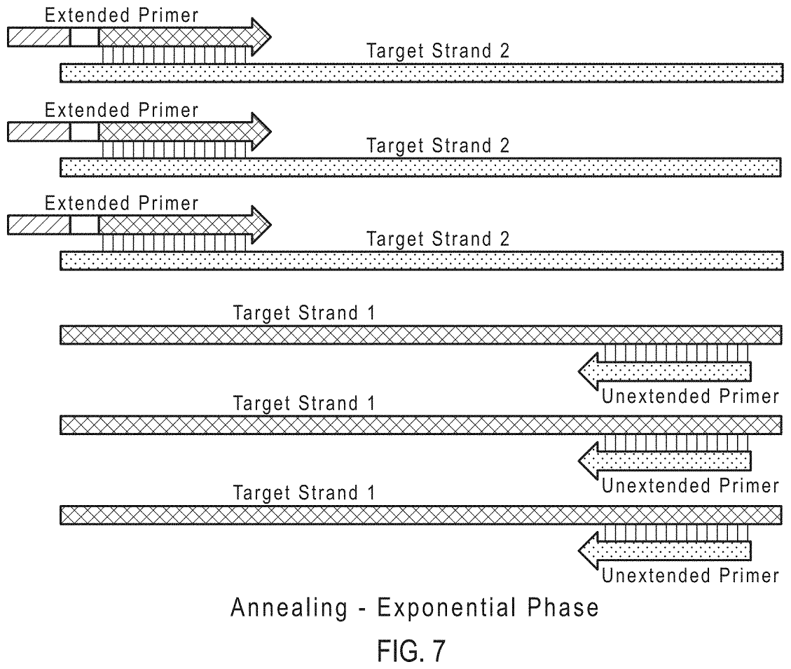

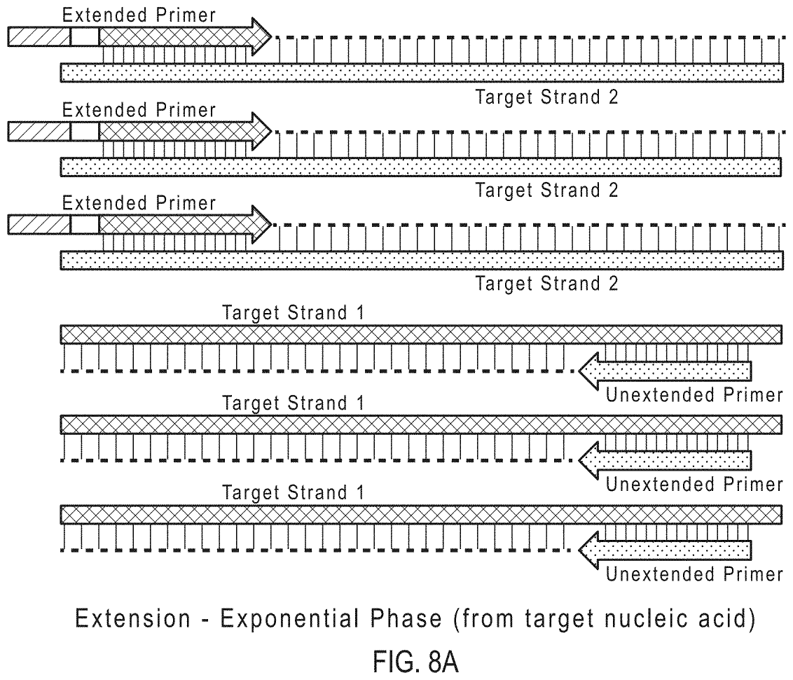

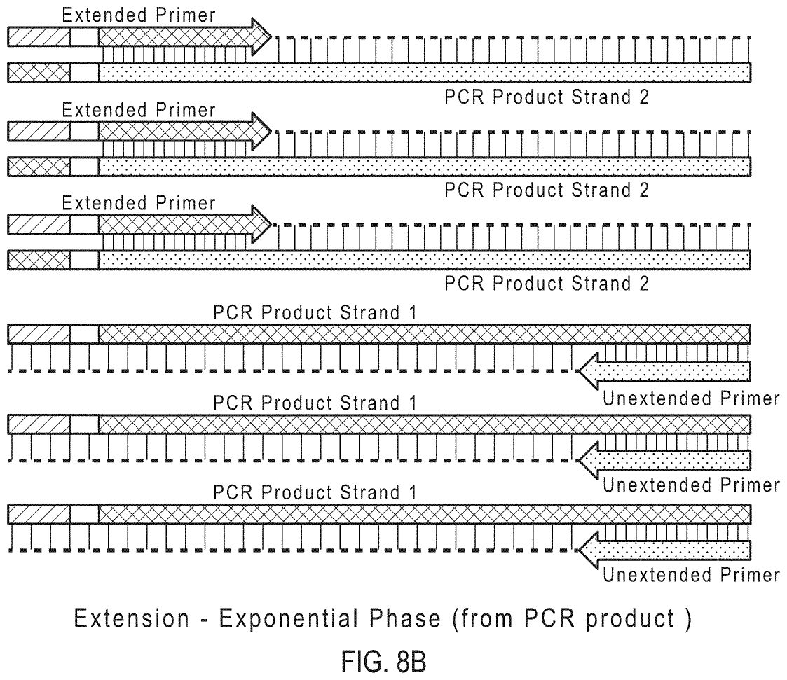

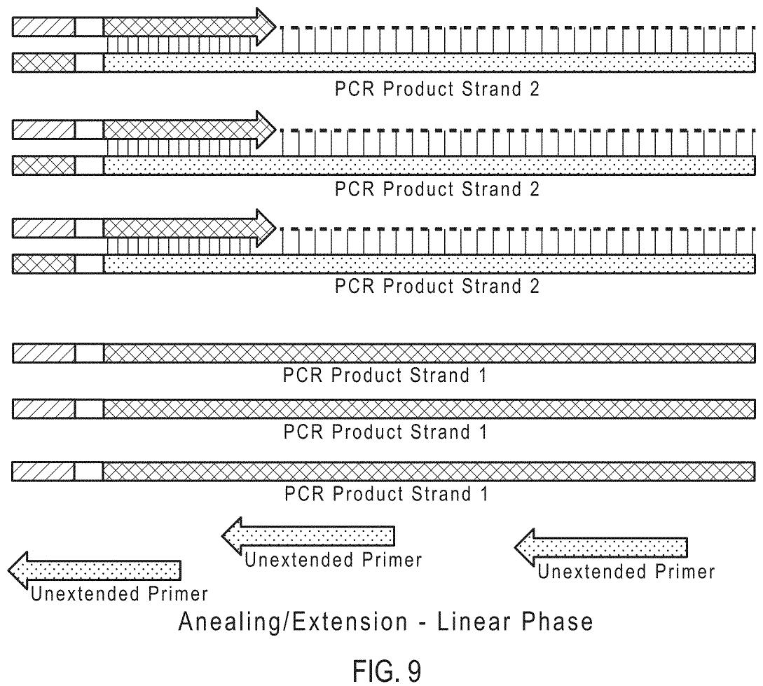

The sequence in the “B” region that is complementary to at least a portion of the “A” region can be a Direct Repeat or Inverted Repeat. Where the “B” region contains a Direct Repeat of a portion of the “A” region, different Extended Primer molecules can hybridize to one another intermolecularly, as shown in FIG. 5B. Where the “B” region contains an Inverted Repeat of a portion of the “A” region, Extended Primer molecules can hybridize intramolecularly, as shown in FIG. 5C, or to one another intermolecularly, as shown in FIG. 5A.

The portion of the “A” region to which a sequence in the “B” region is complementary is preferably at or near (e.g., within 1, 2, or 3 nucleotides from) the 5′ end of the “A” region, i.e., at or near where the “A” region adjoins the “B” region (or the “C” region when a “C” region is present).

The “B” region of the Extended Primer is preferably 6 to 12 nucleotides in length, i.e., is preferably 6, 7, 8, 9, 10, 11 or 12 nucleotides in length. In specific embodiments, the “B” region of the Extended Primer is 8 to 10 nucleotides in length, i.e., is 8, 9 or 10 nucleotides in length.

The “C” region, when present in an Extended Primer, is preferably 1 to 6 nucleotides in length, i.e., is preferably 1, 2, 3, 4, 5, or 6 nucleotides in length.

The Tₘ of the Extended Primer is preferably (but not necessarily) between approximately 68° C. and approximately 80° C. In particular embodiments, the Tₘ of the Unextended Primer is between approximately 72° C. and approximately 78° C., for example approximately 72° C., approximately 73° C., approximately 74° C., approximately 75° C., approximately 76° C., approximately 77° C., or approximately 78° C.

The optional region “C” positioned between regions “A” and “B can act as a spacer between the “A” and “B” regions to allow the Extended Primer to form a hairpin loop and/or introduce a restriction endonuclease sequence (preferably a 6-cutter sequence) into the PCR product. The restriction endonuclease sequence can be within the “C” region in its entirety or be formed from all or a portion of the “C” region together with flanking 5′ and/or 3′ sequences from the “B” and “A” regions, respectively. To minimize interference with hybridization to the target nucleic acid, the “C” region is preferably not complementary to Target Strand 1 or Target Strand 2.

The Tₘ of the Extended Primer is preferably at least approximately 6° C. greater than the Tₘ of the Unextended Primer. Preferably, the Extended Primer has a Tₘ that is at approximately 15° C. to 30° C. greater than the Tₘ of the Unextended Primer.

The Tₘ of the “A” region of the Extended Primer is preferably no more than approximately 3° C. higher or lower than the Tₘ of the portion of the Unextended Primer (at least 75%) complementary to the target (exclusive of any 5′ extensions), i.e., the Tₘ of region in the forward primer that hybridizes to the target is preferably no more than approximately 3° C. higher or lower than the Tₘ of the region in the reverse primer that that hybridizes to the target, and vice versa.

The “A” region of the Extended Primer is preferably at least 12 nucleotides in length, and preferably ranges from 12 to 30 nucleotides and more preferably from 14-25 nucleotides. In certain embodiments, the “A” region of the Extended Primer is 14, 15, 16, 17, 18, 19 or 20 nucleotides in length.

6.2.3.4.2. Unextended Primer

The Unextended Primer has a nucleotide sequence at least 75% sequence identity to a corresponding region in Target Strand 2. In certain embodiments, the Unextended Primer has a nucleotide sequence with at least 80%, at least 85%, at least 90%, or at least 95% sequence identity to the corresponding region in Target Strand 2. In yet other embodiments, the Unextended Primer has a nucleotide sequence with 100% sequence identity to the corresponding region of Target Strand 2.

Stated differently, in various embodiments Unextended Primer has a nucleotide sequence having least 75%, at least 80%, at least 85%, at least 90%, or at least 95% or 100% sequence identity to the complement of the corresponding1 region in Target Strand 2. Typically, the more 5′ any mismatches are between the primer sequence and the target sequence are positioned, the more likely they are to be tolerated during the PCR reaction. One of skill in the art can readily design primer sequences that have less than 100% sequence identity to the target strand but can still efficiently amplify target DNA.

The Unextended Primer may further have a 5′ tail of 1, 2 or 3 nucleotides.

The Tₘ of the Unextended Primer is preferably (but not necessarily) between approximately 50° C. and approximately 62° C. In particular embodiments, the Tₘ of the Unextended Primer is between approximately 59° C. and approximately 62° C., for example approximately 59° C., approximately 60° C., approximately 61° C., or approximately 62° C.

The Tₘ of the Unextended Primer is preferably at least approximately 6° C. lower than the Tₘ of the Extended Primer. Preferably, the Unextended Primer has a Tₘ that is at approximately 15° C. to 30° C. lower than the Tₘ of the Extended Primer.

The Tₘ of the region of the Unextended Primer (at least 75%) complementary to the target (exclusive of any 5′ extensions) is preferably no more than approximately 3° C. higher or lower than the Tₘ of the “A” region of the Extended Primer, i.e., the Tₘ of region in the forward primer that hybridizes to the target is preferably no more than approximately 3° C. higher or lower than the Tₘ of the region in the reverse primer that that hybridizes to the target, and vice versa.

The Unextended Primer is preferably at least 12 nucleotides in length, and preferably ranges from 12 to 30 nucleotides and more preferably from 14-25 nucleotides. In certain embodiments, the Unextended Primer is 14, 15, 16, 17, 18, 19 or 20 nucleotides in length.

6.2.3.4.3. Generic Primer

In some asymmetric PCR methods, for example as described in U.S. Pat. No. 8,735,067 B2, in addition to the forward and reverse primer pair a third, “generic” primer is used that has a sequence that is similar to a 5′ oligonucleotide tail added to one of the primers. The generic primer is intended to participate in the amplification reaction after the initial PCR cycle to “balance” the amplification efficiency of different targets in a multiplex amplification reaction.

Without being bound by theory, it is believed that the inclusion of a generic primer as described in U.S. Pat. No. 8,735,067, which in the context of the improved asymmetric PCR methods would have a sequence consisting essentially of the sequence of the “B” region of the Extended Primer (such generic primers referred to herein as “Generic Primers”), would reduce amplification efficiency using the Asymmetric Primer Pairs described herein. Accordingly, the improved asymmetric DNA amplification methods described herein are preferably performed in the absence of Generic Primers.

In a related embodiment, the improved asymmetric DNA amplification methods described herein can utilize a single Asymmetric Primer Pair per target region, i.e., do not include any additional primers, recognizing that an individual primer may be a mixture of primer molecule with closely related sequences resulting from the inclusion of mixed bases at certain positions in the primer. For clarity and avoidance of doubt, this embodiment does not preclude that use of a plurality of Asymmetric Primer Pairs in a multiplex amplification reaction, provided that a single Asymmetric Primer Pair is used for each amplicon.

### 6.2.3.5. Real-time PCR Amplification

The PCR amplification reaction used in the methods of the disclosure can be a real-time PCR amplification reaction.

Real-time PCR refers to a growing set of techniques in which the buildup of amplified DNA products can be measured as the reaction progresses, typically once per PCR cycle. Monitoring the accumulation of products over time allows for the determination of the efficiency of the reaction, as well as to estimate the initial concentration of DNA template molecules. For general details concerning real-time PCR see Real-Time PCR: An Essential Guide, K. Edwards et al., eds., Horizon Bioscience, Norwich, U.K. (2004).

Several different real-time detection chemistries now exist to indicate the presence of amplified DNA. Most of these depend upon fluorescence indicators that change properties as a result of the PCR process. Among these detection chemistries are DNA binding dyes (such as SYBR® Green) that increase fluorescence efficiency upon binding to double stranded DNA. Other real-time detection chemistries utilize fluorescence resonance energy transfer (FRET), a phenomenon by which the fluorescence efficiency of a dye is strongly dependent on its proximity to another light absorbing moiety or quencher. These dyes and quenchers are typically attached to a DNA sequence-specific probe or primer. Among the FRET-based detection chemistries are hydrolysis probes and conformation probes. Hydrolysis probes (such as the TaqMan® probe) use the polymerase enzyme to cleave a reporter dye molecule from a quencher dye molecule attached to an oligonucleotide probe. Conformation probes (such as molecular beacons) utilize a dye attached to an oligonucleotide, whose fluorescence emission changes upon the conformational change of the oligonucleotide hybridizing to the target DNA (see for example, Tyagi S et al., 1996, Molecular beacons: probes that fluoresce upon hybridization. Nat Biotechnol 14, 303-308).

Real-time PCR can be symmetric or asymmetric, e.g., performed with a hydrolysis probe molecule in the reaction mixture of a symmetric or asymmetric PCR amplification reaction described in Section 6.2.3.3 or 6.2.3.4.

A number of commercial instruments exist that can be used to perform real-time PCR. Examples of available instruments include the Applied Biosystems PRISM 7500, the Bio-Rad iCylcer, and the Roche Diagnostics LightCycler 2.0.

6.2.4. Probe Molecules

The present disclosure provides probe molecules, e.g., oligonucleotide probe molecules, suitable for the sequence specific detection of amplicons produced in a PCR reaction.

Parameters for successful oligonucleotide probe molecule design are well known in the art and include, but are not limited to, probe molecule length, cross-hybridization efficiency, melting temperature, GC-content, self-annealing, and the ability to form secondary structures. The present disclosure provides for the use of oligonucleotide probe molecules in a microarray, e.g., an addressable array, in which the probe molecules are anchored to a substrate, e.g., a membrane, e.g., a glass substrate, e.g., a plastic substrate, e.g., a polymer-matrix substrate, and exposed to nucleic acids under conditions allowing hybridization of the oligonucleotide probe molecule with amplicons having similar to identical sequences, e.g., the sequences share at least 75%, e.g., 80%, e.g., 85%, e.g., 90%, e.g., 95%, e.g., 96%, e.g., 97%, e.g., 98%, e.g., 99%, or even 100% similarity or identity.

In some embodiments, oligonucleotide probe molecules used in the methods of the disclosure comprise a nucleotide sequence that is 90% to 100% complementary (e.g., 90% to 95% or 95% to 100%) to 15 to 40 consecutive nucleotides in a first genomic sequence and/or second genomic sequence.

Exemplary oligonucleotide probe molecules are described in the Examples, and include probe molecules comprising nucleotide sequences of SEQ ID NOs: 1-7.

In some embodiments, the oligonucleotide probe molecules are present on an array. Each probe molecule can be at a discrete at a discrete location on the array and distinguishable by its location on the array such that the oligonucleotide probe molecules are positionally addressable probe molecules present on the array.

In some embodiments, the oligonucleotide probe molecules comprise a poly-thymidine tail, for example, a poly-thymidine tail comprising up to 10 nucleotides, or for example, a poly-thymidine tail comprising up to 15 nucleotides, or, for example, a poly-thymidine tail comprising up to 20 nucleotides. In one embodiment, the poly-thymidine tail comprises 10 to 20 nucleotides, e.g., 15 nucleotides. Poly-thymidine tails can be useful when probe molecules are attached to an array, with the poly-thymidine tail acting as a spacer between the array substrate and the region of the probe molecule having partial or full complementarity to a target sequence(s).

Oligonucleotide probe molecules can be labeled or unlabeled. In some embodiments, oligonucleotide probe molecules are labeled. In other embodiments, oligonucleotide probe molecules are unlabeled. Oligonucleotide probe molecules can be labeled, for example with a fluorescent reporter, which can be a fluorescent dye such as those described in Section 6.2.3 or 6.2.3.1. Labeled oligonucleotide probe molecules can be used, for example, in real-time PCR reactions. Labeled oligonucleotide probe molecules for real-time PCR can comprise a fluorescent reporter at one end of the probe molecule and a quencher moiety at the other end of the probe molecule that quenches fluorescence of the reporter. During PCR, the probe molecule can hybridize to its target sequence during the annealing stage, and once the polymerase reaches the probe molecule during the extension stage, its 5′-3′-exonuclease degrades the probe, physically separating the fluorescent reporter from the quencher, resulting in an increase in fluorescence, which can be measured.

The locations of the fluorescent label and quencher moiety on the probe molecule may be such that FRET may occur between the two moieties. The fluorescent label can be, for example, at or near the 5′ end of the probe molecule and the quencher moiety at or near the 3′ end of the probe. In some embodiments, the separation distance between the fluorescent label and quencher is about 14 to about 22 nucleotides, although other distances, such as from about 6, about 8, about 10, or about 12 nucleotides may be used. Additional distances that can be used include about 14, about 16, about 18, about 20, or about 22 nucleotides.

An exemplary fluorescent label that can be used in an oligonucleotide probe molecule for real-time PCR is FAM or 6-FAM, and a representative quencher moiety is MGB. Other non-limiting examples of a reporter moiety include fluorescein, HEX, TET, TAM, ROX, Cy3, Alexa, and Texas Red while non-limiting examples of a quencher or acceptor fluorescent moiety include TAMRA, BHQ (black hole quencher), LC RED 640, and cyanine dyes such as CY5. As will be appreciated by a person skilled in the art, any pair of reporter and quencher/acceptor moieties may be used as long as they are compatible such that transmission may occur from the donor to the quencher/acceptor. Moreover, pairs of suitable donors and quenchers/acceptors are known in the art and are provided herein. The selection of a pair may be made by any means known in the art. Custom real-time PCR probe molecules are commercially available, for example from ThermoFisher Scientific, Sigma-Aldrich and others.

6.2.5. Virtual Probes

For convenience, this Section (and other sections of the disclosure) refers to amplicons and amplicon sets that can be probed with virtual probes. However, it should be understood that virtual probes can likewise be used to probe samples containing or suspected of containing non-amplified target nucleic acids such as genome fragments.

The number of nucleotide mismatches between a first amplicon set (containing a single first amplicon corresponding to a region in a first genome or a plurality of first amplicons corresponding to different regions in the first genome) and a second amplicon set (containing a single second amplicon corresponding to a region in a second genome or a plurality of second amplicons corresponding to different regions in the second genome) can be relatively small, and individual oligonucleotide probe molecules may not be capable of individually distinguishing between the first and second amplicon sets. The inventors have unexpectedly discovered that it is possible to nevertheless distinguish between the first and second amplicon sets in such situations by using a virtual probe. The probe molecules of the virtual probe cannot individually, but can collectively distinguish between the two amplicon sets by virtue of the different hybridization patterns observed when the first and second amplicon sets are probed with the probe molecules of the virtual probe.

Nucleotide sequences of the first amplicon and the second amplicon should have at least 1 (e.g., 1, at least 2, 2, at least 3, or 3) nucleotide mismatch in the regions of the amplicons capable of being bound by the probe molecules used in a virtual probe so that there is a difference in the signal pattern for the two or more probe molecules that make up the virtual probe when the probe molecules are hybridized to the first amplicon set and when the probe molecules are hybridized to the second amplicon set (e.g., on an array or during a real-time PCR reaction). The difference in the signal pattern can be used to identify and/or distinguish the first and second amplicon sets. When the first amplicon set is determined to be present by use of a virtual probe, it can be concluded that the sample from which the first amplicon set was produced contains the genome corresponding to the first amplicon set (and, by extension, the organism whose genome is contained in the sample). Likewise, when the second amplicon set is determined to be present by use of a virtual probe, it can be concluded that the sample from which the second amplicon set was produced contains the genome corresponding to the second amplicon set (and, by extension, the organism whose genome is contained in the sample).

Signals (e.g., signals that are distinguishable by their location on an array or that correspond to different fluorescent labels) for individual probe molecules of a virtual probe when hybridized to PCR amplification products can be combined, for example, by one or more Boolean operators, by one or more relational operators, or by one or more Boolean operators and one or more relational operators, in any combination, to distinguish between first and second amplicon sets. In some embodiments, the signals are combined by one or more Boolean operators. In other embodiments, the signals are combined by one or more relational operators. In yet other embodiments, the signals are combined by one or more Boolean operators and one or more relational operators.

In some instances, the Boolean operators “AND”, “OR”, and “NOT” can be used to combine the signals from individual probe molecules of a virtual probe to distinguish between a first amplicon set and a second amplicon set. As an example, a virtual probe for two homologous amplicons (“Amplicon A” and “Amplicon B” in this example) consists of two probe molecules (“Probe 1” and “Probe 2” in this example). Both Probe 1 and Probe 2 are capable of specifically hybridizing to Amplicon A, while Probe 1, but not Probe 2, is capable of specifically hybridizing to Amplicon B. When PCR amplification products are probed with the virtual probe and the signal from hybridization of Probe 1 and the signal from hybridization of Probe 2 to the PCR amplification products are both positive (which can be represented using the Boolean operator “AND” as “Probe 1 AND Probe 2”), it can be determined that Amplicon A is present in the PCR amplification products. When PCR amplification products are probed with the virtual probe and the signal from hybridization of Probe 1 to the PCR products is positive, while the signal from hybridization of Probe 2 to the PCR products is not positive (which can be represented using the Boolean operator “NOT” as “Probe 1 NOT Probe 2”), it can be determined that Amplicon B is present in the PCR amplification products. A hybridization signal can be considered positive, for example, if the hybridization signal is above a background level. A hybridization signal can be considered not positive, for example, when no signal is observed or the observed signal is not above a background level.

In some instances, the relational operators “greater than” (“>”) and “less than” (“<”) can be used to combine the signals from individual probe molecules of a virtual probe to distinguish between a first amplicon set and a second amplicon set. As an example, a virtual probe for two homologous amplicons (“Amplicon C” and “Amplicon D” in this example) consists of two probe molecules (“Probe 3” and “Probe 4” in this example). Both Probe 3 and Probe 4 are capable of specifically hybridizing to Amplicon C and Amplicon D. When Probe 3 and Probe 4 are hybridized to Amplicon C, the signal for Probe 3 is greater than the signal for Probe 4 (which can represented using the “greater than” relational operator as “Probe 3>Probe 4”. On the other hand, when Probe 3 and Probe 4 are hybridized to Amplicon D, the signal for Probe 3 is less than the signal for Probe 4 (which can be represented using the “less than” relational operator as “Probe 3<Probe 4”). Thus, when PCR amplification products are probed with the virtual probe and the signal for Probe 3 is grater than the signal for Probe 4, it can be determined that Amplicon C is present in the PCR amplification products, and when the signal for Probe 3 is less than the signal for Probe 4, it can be determined that Amplicon D is present in the PCR amplification products.

When combining hybridization signals, the signals can be, for example, absolute signals, normalized signals, or fractional signals (e.g., the value of a signal for a probe molecule used in a virtual probe can be scaled using a predetermined function, for example as described in Example 3). A signal for a probe molecule can be considered positive, for example, when it is above a predetermined cut-off. A cut-off can be, for example, set at or above a background signal observed for a given probe molecule (e.g., a background signal due to non-specific hybridizing). Thus, for example, if a signal for a probe molecule is observed, but the signal is not above a background level, the signal can be considered not positive.

In one embodiment, a virtual probe comprises two or more oligonucleotide probe molecules (e.g., 2 oligonucleotide probe molecules). In another embodiment, a virtual probe comprises three or more oligonucleotide probe molecules (e.g., 3 oligonucleotide probe molecules or 4 oligonucleotide probe molecules).

In some embodiments, a virtual probe for a first organism and a second organism consists of two probe molecules. In one embodiment, the two probe molecules comprise a first probe molecule capable of specifically hybridizing a first amplicon in a first amplicon set (corresponding to the first organism) and a second amplicon in a second amplicon set (corresponding to the second organism), and a second probe molecule that is capable of specifically hybridizing to an amplicon in the second amplicon set but not an amplicon in the first amplicon set. In such an embodiment, it can be determined that the first organism is present in a sample if the signal for the first probe molecule is positive and the signal for the second probe molecule is not positive when probing PCR amplification products prepared from the sample. On the other hand, if the signal for the first probe molecule is positive and the signal for the second probe molecule is positive when probing the PCR amplification products it can be determined that the second organism is present in the sample.

In some embodiments, a virtual probe for a first organism and a second organism consists of three probe molecules. In one embodiment, the three probe molecules comprise a first probe molecule capable of specifically hybridizing a first amplicon in a first amplicon set (corresponding to the first organism) and a second amplicon in a second amplicon set (corresponding to the second organism), a second probe molecule capable of specifically hybridizing to an amplicon in the first amplicon set and an amplicon in the second amplicon set, and which is different from the first probe, and a third probe molecule capable of specifically hybridizing to an amplicon in the first amplicon set and an amplicon in the second amplicon set, and which is different from the first and second probe molecules. In such embodiments, the relative signals for the three probe molecules observed when probing a PCR amplification product can be used to determine whether the sample used to prepare the PCR amplification products contains the first organism or the second organism.

Because virtual probes can be used to distinguish homologous genomic sequences, virtual probes can be used to distinguish closely related organisms, for example closely related microorganisms. For example, virtual probes can be used to distinguish microorganisms from the same order, the same family, the same genus, the same group, or even the same species (e.g., different strains of the same species). For example, virtual probes can be used to distinguish between Lactobacillus and Listeria species, distinguish between Corynebacterium and Propionibactium species, distinguish between Micrococcus and Kocuria species, distinguish between Pasteurella and Haemophillus species, distinguish between coagulase negative Staphylococcus species and coagulase positive Staphylococcus species, distinguish Streptococcus species (e.g., S. anginosus, S. gordonii, S. mitis, S. pneumoniae, S. agalactiae, S. pyogenes. S. gallolyticus, S. infantarius, S. vestibularis, S. salivarius, S. hyointestinalis, S. constellatus, S. intermedius, S. oralis, S. sanguinis, S. parasanguinis), distinguish Staphylococcus species (e.g., S. lugdunensis, S. epidermidis), distinguish Enterococcus species (e.g., E. fecalis, E. faecium), distinguish Clostridium species (e.g., C. perfringens, C. clostridiiforme, C. innocuum), distinguish Bacillus species (e.g., B. cereus, B. coagulans), distinguish Pseudomonas species (e.g., P. aeruginosa, P. putida, P. stutzeri, P. fluorescens, P. mendocina), and distinguish Acinetobacter species (e.g., A. baumannii, A. Iwoffii, A. ursingii, A. haemolyticus, A. junii).

Sections 6.2.5.1 to 6.2.5.3 and Examples 1-5 in Section 7 describe exemplary virtual probes for identifying and/or distinguishing different types of closely related bacteria.

6.2.5.1. Virtual Probes for Coagulase Negative Staphylococcus sp.

The disclosure provides virtual probes that can be used to determine if a coagulase negative Staphylococcus sp. is present in a sample, and that can be used to distinguish a sample comprising a coagulase negative Staphylococcus sp. from a sample comprising a coagulase positive Staphylococcus sp. A first exemplary probe molecule that can be used in a virtual probe for coagulase negative Staphylococcus sp. comprises or consists of the nucleotide sequence CCAGTCTTATAGGTAGGTTAYCCACG (SEQ ID NO:1). A second exemplary probe molecule that can be used in a virtual probe for coagulase negative Staphylococcus sp. comprises or consists of the nucleotide sequence GCTTCTCGTCCGTTCGCTCG (SEQ ID NO:2). The nucleotide sequences of SEQ ID NO:1 and SEQ ID NO:2 are designed to probe 16S RNA amplicons. Thus, probe molecules having nucleotide sequences of SEQ ID NO:1 or SEQ ID NO:2 can be used to probe amplicons produced by a PCR amplification reaction performed using primers designed to amplify coagulase negative Staphylococcus sp. 16S rRNA genomic sequences. The probe molecules can be, for example, included on an array or used in a real-time PCR reaction.

It can be determined that a sample contains a coagulase negative Staphylococcus sp. if, when probing PCR amplification products prepared from the sample, the signal for the first oligonucleotide probe (“Probe 1”) is positive and the signal for the second oligonucleotide probe (“Probe 2”) is not positive (which can be represented using the “NOT” operator as “Probe 1 NOT Probe 2”). Exemplary virtual probes for coagulase negative Staphylococcus sp. are further described in Example 1.

### 6.2.5.2. Virtual Probes for Streptococcus gordonii and Streptococcus anginosus

The disclosure provides virtual probes that can be used to determine if Streptococcus gordonii or Streptococcus anginosus is present in a sample, and that can be used to distinguish a sample comprising Streptococcus gordonii from a sample comprising Streptococcus anginosus. A first exemplary probe molecule that can be used in a virtual probe for S. gordonii and S. anginosus comprises or consists of the nucleotide sequence CAGTCTATGGTGTAGCAAGCTACGGTAT (SEQ ID NO:3). A second exemplary probe molecule that can be used in a virtual probe for S. gordonii and S. anginosus comprises or consists of the nucleotide sequence TATCCCCCTCTAATAGGCAGGTTA (SEQ ID NO:4). The nucleotide sequences of SEQ ID NO:3 and SEQ ID NO:4 are designed to probe 16S RNA amplicons. Thus, oligonucleotide probe molecules having nucleotide sequences of SEQ ID NO:3 or SEQ ID NO:4 can be used to probe amplicons produced by a PCR amplification reaction performed using primers designed to amplify 16S rRNA genomic sequences from S. gordonii and S. anginosus. The probe molecules can be, for example, included on an array or used in a real-time PCR reaction.

It can be determined that a sample contains S. gordonii if, when probing PCR amplification products prepared from the sample, the signal for the first probe (“Probe 1”) is positive and the signal for the second probe (“Probe 2”) is not positive (which can be represented using the “NOT” operator as “Probe 1 NOT Probe 2”). It can be determined that a sample contains S. anginosus if, when probing PCR amplification products prepared from the sample, the signal for Probe 1 is positive and the signal for Probe 2 is also positive (which can be represented using the “AND” operator as “Probe 1 AND Probe 2”). Exemplary virtual probes for S. gordonii and S. anginosus are further described in Example 2.

### 6.2.5.3. Virtual Probes for Streptococcus mitis and Streptococcus pneumoniae

The disclosure virtual probes that can be used to determine if Streptococcus mitis or Streptococcus pneumoniae is present in a sample, and that can be used to distinguish a sample comprising Streptococcus mitis from a sample comprising Streptococcus pneumoniae. A first exemplary probe molecule that can be used in a virtual probe for S. mitis and S. pneumoniae comprises or consists of the nucleotide sequence AGCTAATACAACGCAGGTCCATCT (SEQ ID NO:5). A second exemplary probe molecule that can be used in a virtual probe for S. mitis and S. pneumoniae comprises or consists of the nucleotide sequence GATGCAAGTGCACCTTTTAAGCAA (SEQ ID NO:6). A third exemplary probe molecule that can be used in a virtual probe for S. mitis and S. pneumoniae comprises or consists of the nucleotide sequence GATGCAAGTGCACCTTTTAAGTAA (SEQ ID NO:7). The nucleotide sequences of SEQ ID NO:5, SEQ ID NO:6, and SEQ ID NO:7 are designed to probe 16S RNA amplicons. Thus, probe molecules having nucleotide sequences of SEQ ID NO:5, SEQ ID NO:6, or SEQ ID NO:7 can be used to probe amplicons produced by a PCR amplification reaction performed using primers designed to amplify 16S rRNA genomic sequences of S. mitis and S. pneumoniae. The probe molecules can be, for example, included on an array or used in a real-time PCR reaction.

It can be determined that a sample contains S. mitis if, when probing PCR amplification products prepared from the sample, the signals for the second probe (“Probe 2”) and/or third probe (“Probe 3”) is/are less than a scaled signal for the first probe (“Probe 1”). The relationship between the signals for determining whether the sample contains S. mitis can be represented using Boolean and relational operators as “(Probe 2 OR Probe 3)<(Probe 1)/n,” where n is a pre-determined value used to scale the Probe 1 signal. It can be determined that a sample contains S. pneumoniae if, when probing PCR amplification products prepared from the sample, the signal for Probe 2 and/or Probe 3 is/are greater than a scaled signal for Probe 1. The relationship between the signals for determining whether the sample contains S. pneumoniae can be represented using Boolean and relational operators as “(Probe 2 OR Probe 3)>(Probe 1)/n.” A suitable value for “n” can be determined, for example, by probing PCR products produced from a sample known to contain S. mitis and probing PCR products from a sample known to contain S. pneumoniae.

Alternatively, it can be determined that a sample contains S. mitis if, when probing PCR amplification products prepared from the sample, the signal for Probe 3 divided by the signal for Probe 1 is less than a predetermined value “n”. It can be determined that a sample contains S. pneumoniae if, when probing PCR amplification products prepared from the sample, the signal for Probe 3 divided by the signal for probe 1 is greater than “n”. A suitable value for “n” can be determined, for example, by probing PCR products produced from a sample known to contain S. mitis and probing PCR products from a sample known to contain S. pneumoniae.

Exemplary virtual probes for S. mitis and S. pneumoniae are further described in Example 3.

6.3. Arrays

The present disclosure provides addressable arrays comprising one or more virtual probes that each can be useful for distinguishing a first genomic sequence from a second, homologous genomic sequence.

The addressable arrays of the disclosure can be used in the methods described herein. An addressable array of the disclosure can comprise a group of positionally addressable oligonucleotide probe molecules, each at a discrete location on the array. In some embodiments, each probe molecule in the group of oligonucleotide probe molecules making up a virtual probe (typically two or three different probe molecules) comprises a nucleotide sequence that is 90% to 100% (e.g., 90% to 95% or 95% to 100%) complementary to 15 to 40 consecutive nucleotides (e.g., 15 to 20, 15 to 30, 20 to 40, 20 to 30, or 30 to 40 consecutive nucleotides) in the first genomic sequence or second genomic sequence that the virtual probe is intended to distinguish.

The addressable array may further optionally comprise one or more control probe molecules (e.g., an extraction and amplification control for useful for evaluating the efficiency of DNA extraction and amplification steps and/or a hybridization control useful for evaluating the efficiency of DNA hybridization to the array).

In some embodiments, the probe molecules of the array comprise a poly-thymidine tail, for example, a poly-thymidine tail comprising up to 10 nucleotides, or for example, a poly-thymidine tail comprising up to 15 nucleotides, or, for example, a poly-thymidine tail comprising up to 20 nucleotides. In some embodiments, the poly-thymidine tail is a 10-mer to a 20-mer, e.g., a 15 mer.

In some embodiments, the addressable array comprises 12 or more probe molecules, for example, 12 to 100 probe molecules, or for example, 12 to 50 probe molecules, or for example, 25 to 75 probe molecules, or for example, 50 to 100 probe molecules. In some embodiments, the addressable array comprises 12 probe molecules. In other embodiments, the addressable array comprises 14 probe molecules. In still other embodiments, the addressable array comprises 84 probe molecules.

In some embodiments, the addressable array comprises oligonucleotide probes for at least 2 virtual probes, for example, for at least 3 virtual probes, or for example, at least 5 virtual probes, or for example at least 10 virtual probes, or for example, the addressable array comprises oligonucleotide probes for up to 10 or up to 15 virtual probes.

The virtual probes can be overlapping such that a probe molecule can be a component of two or more virtual probes. The virtual probes can also be non-overlapping.

In some embodiments, the addressable array comprises virtual probes capable of distinguishing between at least 5 different types of microorganisms, for example bacteria. In other embodiments, the addressable array comprises virtual probes capable of distinguishing between at least 10 different types, for example, at least 20 different types, for example at least 30 different types, for example, at least 40 different types, or for example up to 50 different types of microorganisms, for example bacteria.

In some embodiments, the addressable array contains at least 5 virtual probes, for example, at least 10 virtual probes, for example at least 15 virtual probes, or for example, at least 20 virtual probes, each of which is capable of identifying different types of microorganisms, for example bacteria, for example, different strains or species of bacteria that might be present in a sample.

In some embodiments, an addressable array of the disclosure comprises one or more virtual probes for differentiating a genomic sequence from a species of eubacteria from a genomic sequence of a microorganism that is not a species of eubacteria. In some embodiments, the addressable array comprises one or more virtual probes suitable for differentiating a genomic sequence from a gram positive bacteria and a genomic sequence from a gram negative bacteria. In some embodiments, the addressable array comprises one or more virtual probes suitable for differentiating genomic sequences from microorganisms of different orders. In some embodiments, the virtual probes are suitable for differentiating genomic sequences from microorganisms of different families. In some embodiments, the virtual probes are suitable for differentiating genomic sequences from microorganisms of different genera, of different groups, and/or of different species.

Suitable microarray system that can be used to make an array of the disclosure are described in U.S. Pat. No. 9,738,926 and U.S. Patent Application Publication no. 2018/0362719 A1, the contents of which are incorporated by reference herein in their entireties. The microarray systems described in U.S. Pat. No. 9,738,926 and U.S. Patent Application Publication no. 2018/0362719 A1 take advantage of three-dimensional crosslinked polymer networks. Thus, in some embodiments, the array of the disclosure comprises an array as described in U.S. Pat. No. 9,738,926, wherein the probe molecules of the array comprise a group of oligonucleotide probe molecules as described herein. In other embodiments, the array of the disclosure comprises an array as described in U.S. Patent Application Publication no. 2018/0362719 A1, wherein the probe molecules of the array comprise a group of oligonucleotide probe molecules as described herein.

In one aspect of the disclosure, the present disclosure provides methods for determining if a first organism or a second organism is present in a sample using an array of the disclosure. An exemplary method comprises the steps of:
performing a PCR amplification reaction on a sample using PCR primers capable of hybridizing to, and initiating a PCR amplification from, both the genome of the first organism (the “first genome”) and the genome of the second organism (the “second genome”), resulting in a first amplicon set and a second amplicon set, respectively, when the first genome and the second genome are present in the sample, and wherein the PCR amplification reaction incorporates a label which produces a measurable signal into any PCR amplification products produced by the reaction;
contacting the PCR amplification products to an array of the disclosure having one or more virtual probes comprising two or more oligonucleotide probe molecules each of which is capable of specifically hybridizing to one or more amplicons in the first amplicon set and/or the second amplicon set, and wherein the two or more oligonucleotide probe molecules hybridize non-identically to the amplicons in the first amplicon set and the amplicons in the second amplicon set, such that the hybridizing of the probe molecules to the amplicons in the first amplicon set and the second amplicon set can distinguish between the first amplicon set and the second amplicon set;
washing unbound nucleic acid molecules from the array; and
measuring the signal of the label at each probe molecule location on the array; and
if the signals indicate that PCR amplification products that hybridize to the probe molecules are produced by the PCR amplification reaction, analyzing the signals as described herein to determine if the first amplicon set or second amplicon set is produced by the PCR amplification reaction; or if the signals indicate that no PCR amplification products that hybridize to the group of probe molecules are produced by the PCR amplification reaction, determining that that sample does not contain the first organism or the second organism,
thus determining if the first organism or second organism is present in the sample.

6.4. Systems

The present disclosure provides systems for determining if an organism is present in a sample. The systems can comprise, for example: (i) an optical reader for generating signal data for each probe molecule location of an array having oligonucleotide probe molecules (e.g., an array of the disclosure); and (ii) at least one processor which is configured to receive signal data from the optical reader and is configured to analyze the signal data using a virtual probe (e.g., a virtual probe having features as described herein), and which has an interface to a storage or display device or network for outputting a result of the analysis.

Optical readers that can be used in the systems of the disclosure include commercially available microplate readers (e.g., GloMax Discover (Promega), ArrayPix™ (Arrayit), Varioskan™ LUX (Thermo Scientific), Infinite® 200 PRO (Tecan)).

The system can include a non-transient storage medium (e.g., a hard disk, flash drive, CD or DVD) including processor executable instructions for implementing the analysis of the signal data.

The system can include a general purpose or a special purpose computing system environment or configuration. Examples of well-known computing systems, environments, and/or configurations that can be used with the systems of the disclosure include, but are not limited to, personal computers, server computers, smartphones, tablets, hand-held or laptop devices, multiprocessor systems, microprocessor-based systems, network PCs, minicomputers, mainframe computers, distributed computing environments that include any of the above systems or devices, and the like.

Systems of the disclosure can execute computer-executable instructions, such as program modules. Generally, program modules include routines, programs, objects, components, data structures, etc. that perform particular tasks or implement particular abstract data types. Some embodiments may also be practiced in distributed computing environments where tasks are performed by remote processing devices that are linked through a communications network. These distributed systems may be what are known as enterprise computing systems or, in some embodiments, may be “cloud” computing systems. In a distributed computing environment, program modules may be located in both local and/or remote computer storage media including memory storage devices.

A computing environment may include one or more input/output devices. Some such input/out devices may provide a user interface. A user may enter commands and information into the computer through input devices such as a keyboard and pointing device, such as a mouse. However, other forms of pointing devices may be used, including a trackball, touch pad or touch screen.

Systems of the disclosure can include one or more output devices, including an output device that may form a portion of a user interface, for example a monitor.

Systems of the disclosure can be operated in a networked environment using logical connections to one or more remote computers. The remote computer may be a personal computer, a server, a router, a network PC, a peer device or other common network node.

Logical connections include a local area network (LAN) and a wide area network (WAN), but may also include other networks. Such networking environments are commonplace in offices, enterprise-wide computer networks, intranets and the Internet. Alternatively or additionally, the WAN may include a cellular network.

When used in a LAN networking environment, a system of the disclosure can be connected to the LAN through a network interface or adapter. When used in a WAN networking environment, a system can include a modem or other means for establishing communications over the WAN, such as the Internet.

In a networked environment, program modules for analyzing signal data using a virtual probe can be stored in a remote memory storage device (e.g., hard drive or flash drive).

A system of the disclosure can further comprise a plate handling robot capable of adding the product of a PCR amplification reaction to the array and capable of washing unbound nucleic acid molecules from the array. Numerous plate handling robots are commercially available and such robots can be used in the systems of the disclosure (e.g., a Tecan MSP 9000, MSP 9250 or MSP 9500, a Tecan Cavro® Omni Flex, a Tricontinent TriTon (XYZ), or an Aurora Versa™)

6.5. Kits

The present disclosure provides kits suitable for use in the methods of the disclosure.

A kit can comprise, for example, a set of two or more labeled probe molecules (e.g., 2 to 20 probe molecules, 2 to 10 probe molecules, 2 to 5 probe molecules, 5 to 10 probe molecules, or 10 to 20 probe molecules) suitable for use in a real-time PCR reaction as described herein. For example, a kit can comprise (1) a probe molecule whose nucleotide sequences comprises SEQ ID NO:1 and a probe molecule whose nucleotide sequence comprises SEQ ID NO:2; (2) a probe molecule whose nucleotide sequences comprises SEQ ID NO:3 and a probe molecule whose nucleotide sequence comprises SEQ ID NO:4; or (3) a probe molecule whose nucleotide sequences comprises SEQ ID NO:5, a probe molecule whose nucleotide sequence comprises SEQ ID NO:6, and a probe molecule whose nucleotide sequence comprises SEQ ID NO:7. In some embodiments, a kit comprises a combination of the probe molecules of (1) and (2). In other embodiments, a kit comprises a combination of the probe molecules of (1) and (3). In other embodiments, a kit comprises a combination of the probe molecules of (2) and (3). In yet other embodiments, a kit comprises a combination of the probe molecules of (1), (2), and (3).

In other embodiments, a kit can comprise, for example, a set of two or more probe molecules (e.g., 2 to 20 probe molecules, 2 to 10 probe molecules, 2 to 5 probe molecules, 5 to 10 probe molecules, or 10 to 20 probe molecules) suitable for use on an array as described herein (e.g., unlabeled probe molecules). For example, a kit can comprise (1) a probe molecule whose nucleotide sequences comprises SEQ ID NO:1 and a probe molecule whose nucleotide sequence comprises SEQ ID NO:2; (2) a probe molecule whose nucleotide sequences comprises SEQ ID NO:3 and a probe molecule whose nucleotide sequence comprises SEQ ID NO:4; or (3) a probe molecule whose nucleotide sequences comprises SEQ ID NO:5, a probe molecule whose nucleotide sequence comprises SEQ ID NO:6, and a probe molecule whose nucleotide sequence comprises SEQ ID NO:7. In some embodiments, a kit comprises a combination of the probe molecules of (1) and (2). In other embodiments, a kit comprises a combination of the probe molecules of (1) and (3). In other embodiments, a kit comprises a combination of the probe molecules of (2) and (3). In yet other embodiments, a kit comprises a combination of the probe molecules of (1), (2), and (3).

In other embodiments, a kit can comprise an array as described herein.

Kits as described herein can further comprise one or more reagents for performing a PCR reaction e.g., one or more (e.g., two) primers for amplifying homologous genomic sequences, and/or one or more reagents for performing a hybridization reaction, e.g., a wash buffer.

Kits as described herein can further comprise one or more reagents and/or one or more devices for preparing a sample for a PCR amplification reaction, e.g., a lysis buffer or a bead beating system.

Kits as described herein can further comprise one or more containers and/or instructions for using the components of the kit to perform some or all of the steps of a method as described herein.

### 7. EXAMPLES

### 7.1. Example 1: Virtual Probes for Coagulase Negative Staphylococci (CNS)

Staphylococcus aureus is a coagulase positive species and is a normal member of the microbiota of the body. However, S. aureus can become an opportunistic pathogen, causing skin infections, respiratory infections, and food poisoning. Thus, there is a clinical need for tests which can distinguish between S. aureus and other Staphylococcus species in clinical samples. There are a few other coagulase positive Staphylococci, but they usually play no major role in disease and therefore can be neglected for most analytical purposes.

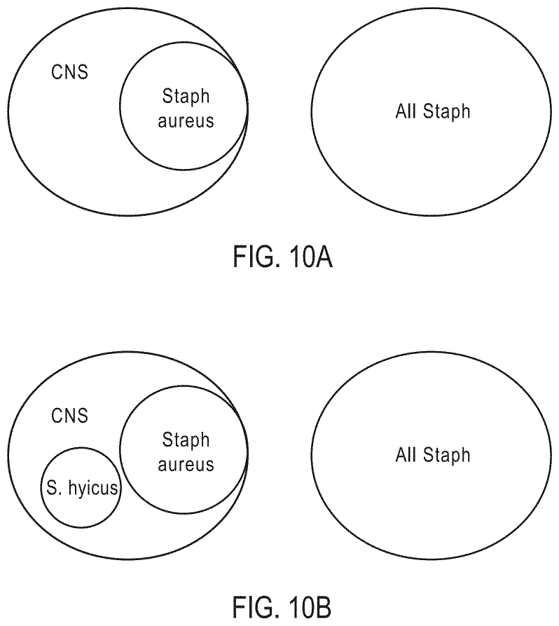

An oligonucleotide probe, “AllStaph-146abp” (having the nucleotide sequence CCAGTCTTATAGGTAGGTTAYCCACG (SEQ ID NO:1)), was made that can be used to non-specifically identify Staphylococcus species. In other words, AllStaph-146abp is a genus probe molecule and cannot by itself distinguish S. aureus from coagulase negative species in a sample. The numbers present in the probe molecule names used in the examples refer to the distance in number of nucleotides between a forward PCR primer for making an amplicon that can be probed with the probe molecule and the start of the probe. A second oligonucleotide probe, “Sau-71p” (having the nucleotide sequence GCTTCTCGTCCGTTCGCTCG (SEQ ID NO:2)) is a 16S rRNA probe molecule that provides a positive signal with amplicons from S. aureus but does not provide a positive signal with amplicons from coagulase negative Staphylococcus. Thus, where the only clinically relevant coagulase positive Staphylococcus species is S. aureus, an exemplary virtual probe for coagulase negative Staphylococcus species can consist of AllStaph-146abp and Sau-71p. When probing a PCR amplification product with the virtual probe and the signal for AllStaph-146abp is positive and the signal for Sau-71p is not positive (which can be represented as “AllStaph-146-abp NOT Sau-71p”), it can be determined that the sample from which the PCR amplification product was made contains a coagulase negative Staphylococcus species (see, FIG. 10A). In a situation where more species are relevant, the virtual probe can include additional probe molecules. For example, when S. hyicus, which can cause skin disease in cattle, horses, and pigs, is relevant, the sample can be determined to contain a coagulase negative Staphylococcus species when a probe specific for S. hyicus is also not positive (which can be represented as “AllStaph-146abp NOT Sau71P NOT S. hyicus”) (see, FIG. 10B).

### 7.2. Example 2: Virtual Probes for Differentiating Streptococcus anginosus and Streptococcus gordonii

Streptococcus gordonii is a bacterium normally found in the human mouth. S. gordonii is usually harmless in the mouth, but can cause acute bacterial endocarditis upon entry to the bloodstream. Streptococcus anginosus is also a member of the human microbiota, and is known to cause infections in immunocompromised individuals.

Two oligonucleotide probe molecules have been made that can be used in virtual probes for distinguishing between S. anginosus and S. gordonii in a sample. The oligonucleotide probe molecule “Stango 85p,” having the nucleotide sequence CAGTCTATGGTGTAGCAAGCTACGGTAT (SEQ ID NO:3), is a 16S rRNA probe molecule which can give a positive signal when either of S. anginosus and S. gordonii are present in a sample. The oligonucleotide probe molecule “Sang 156p,” having the nucleotide sequence TATCCCCCTCTAATAGGCAGGTTA (SEQ ID NO:4), on the other hand, provides a positive signal with amplicons from S. anginosus and does not provide a positive signal with amplicons from S. gordonii. An exemplary virtual probe for S. gordonii and S. anginosus consists of Stango85p and Sang156p. When probing a PCR amplification product with the virtual probe and the signal for Stango85p is positive and the signal for San156p is not positive (which can be represented as “Stango85p NOT Sang156p”) it can be determined that the sample used to prepare the PCR amplification product contains S. gordonii, while if the signal for Stango85p is positive and the signal for San156p is positive (which can be represented as “Stango85p AND Sang156p”), it can be determined that the sample contains S. anginosus.

### 7.3. Example 3: Virtual Probes for Differentiating Streptococcus mitis and Streptococcus pneumoniae

Streptococcus mitis and Streptococcus pneumoniae, both of which can be pathogenic, are almost identical in their 16S rRNA, thereby making it difficult to distinguish between the two species using single oligonucleotide probe molecules for 16S rRNA.

Three oligonucleotide probe molecules have been made that can be used in virtual probes for distinguishing between S. mitis and S. pneumoniae. The first probe, “AllStrep-261p,” having the nucleotide sequence AGCTAATACAACGCAGGTCCATCT (SEQ ID NO:5), is a genus probe molecule that cannot distinguish between different Streptococci species. The second probe, “Spneu-229p,” having the nucleotide sequence GATGCAAGTGCACCTTTTAAGCAA (SEQ ID NO:6), although comprising a genomic sequence from S. pneumoniae, cannot by itself be used to distinguish between S. mitis and S. pneumoniae. The third probe, “Spneu-229 bp,” having the nucleotide sequence GATGCAAGTGCACCTTTTAAGTAA (SEQ ID NO:7), differs from Spneu-229p by a single nucleotide to account for a SNP in S. pneumoniae.

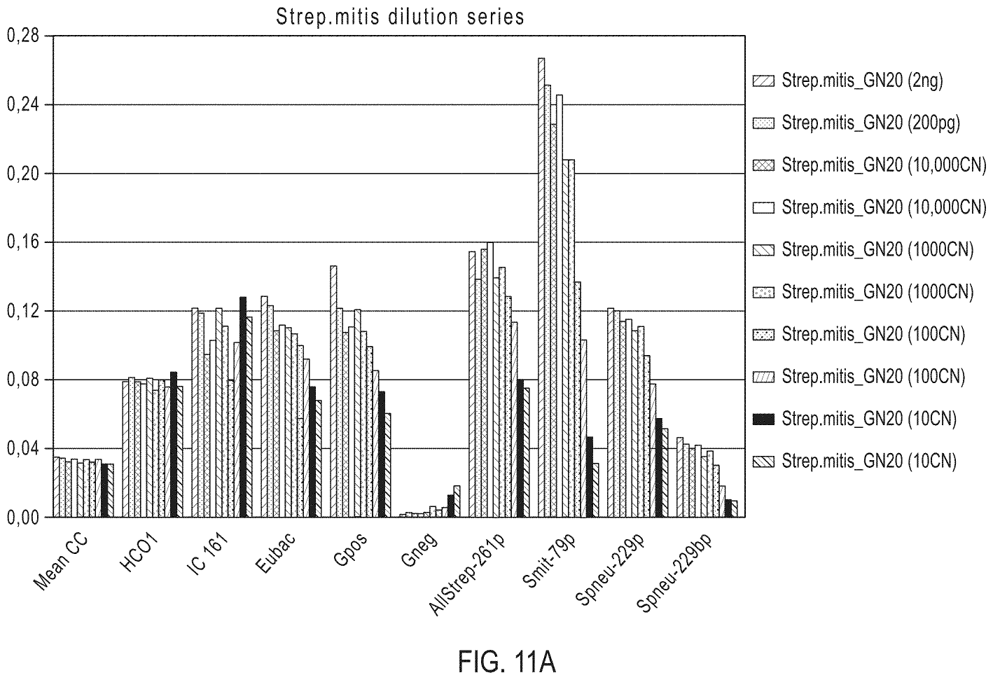

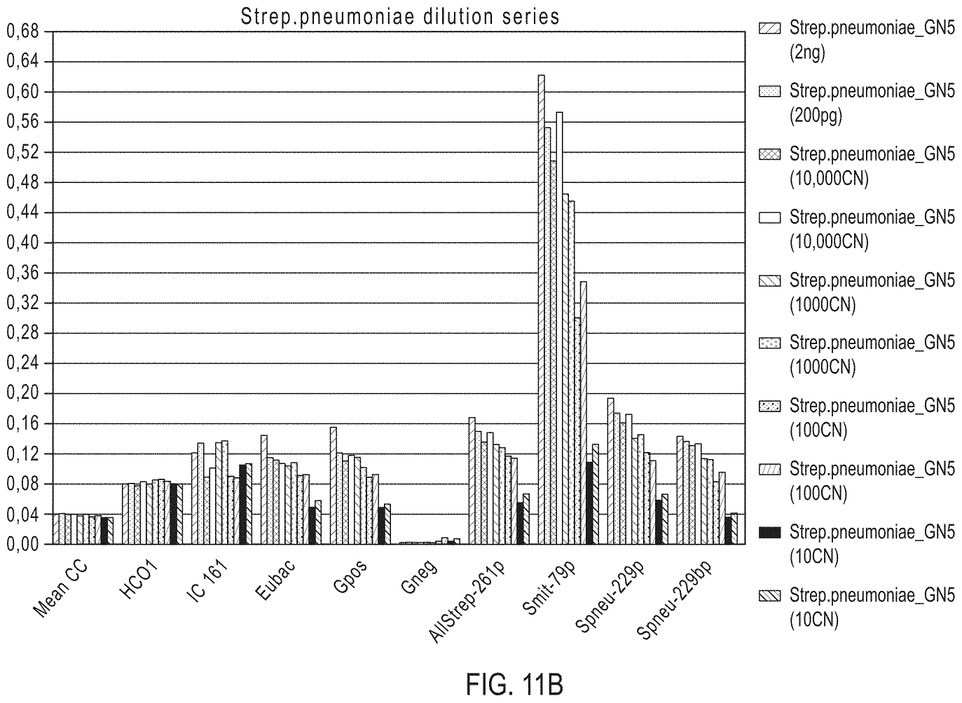

Positive signals for each of the three probe molecules were observed when 16S rRNA amplicons from S. mitis and S. pneumoniae were bound to arrays comprising the three probe molecules (FIG. 11A-11B). Thus, the three probe molecules cannot individually be used to distinguish between S. mitis and S. pneumoniae. However, amplicons from S. mitis and S. pneumoniae could be distinguished by evaluating the signal pattern when probing 16S rRNA amplicons from S. mitis and S. pneumoniae with the three probes. Specifically, probing PCR amplification products produced from a sample containing S. mitis produced a signal pattern that can be represented as “(Spneu-229p OR Spneu-229 bp)<(AllStrep-261p)/3,” while probing PCR amplification products produced from a sample containing S. pneumoniae produced a signal pattern that can be represented as “(Spneu-229p AND Spneu-229 bp)>(AllStrep-261p)/3”.

From further analysis of hybridization data from S. mitis and S. pneumoniae containing samples, it was determined that a signal pattern for the Spneu-229 bp and AllStrep-261p probes of “(Spneu-229 bp/AllStrep-261p) 50.39” indicates the presence of S. mitis, while a signal pattern for the Spneu-229 bp and AllStrep-261p probes of “(Spneu-229 bp/AllStrep-261p)>0.39” indicates the presence of S. pneumoniae.

Thus, this example validates the virtual probe concept.

### 7.4. Example 4: Virtual Probe for Detecting Streptococcus viridians Group

The viridians Streptococci group (VGS) is one of the major group of clinically relevant gram positive bacteria with over 24 species which are arranged in five sub-groups, the Streptococcus bovis group, Streptococcus anginosus group, Streptococcus salivarius group, Streptococcus mitis group and the Streptococcus mutans group. VGS group bacteria can cause pneumonia and sepsis in immunocompromised patients.

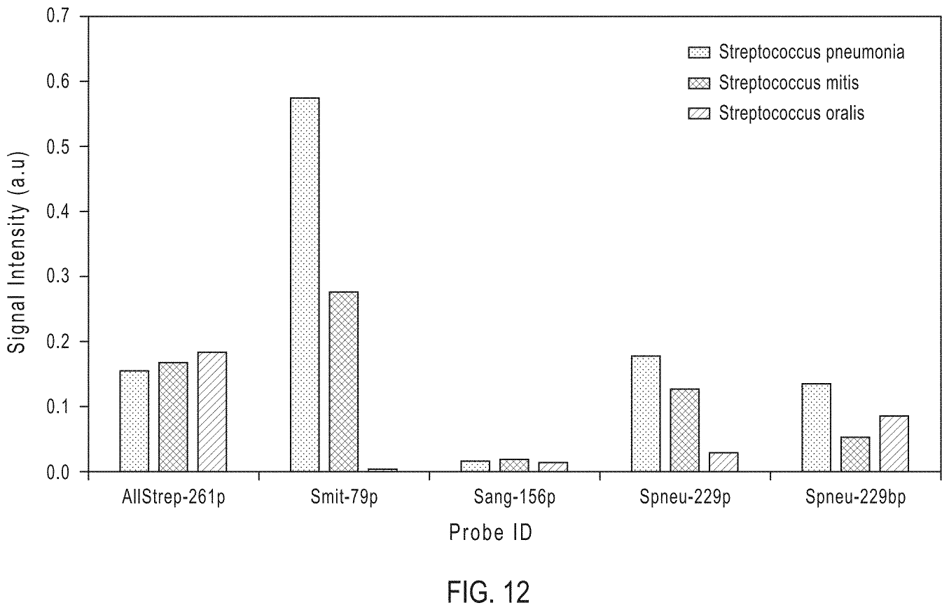

The VGS species as a group appear genetically heterogeneous, suggesting that different species can be detected with a single probe. Multiple probes were designed for the different VGS group bacteria, but a few of the species showed cross-reactivity with probes for other VGS sub-groups (see, FIG. 12, which shows cross-reactivity of S. pneumoniae, with S. mitis probe Smit-79p, and which shows S. mitis and S. oralis cross-reactivity with S. pneumoniae probes Spneu-229p and Spneu-229 bp). In view of the cross-reactivity, a virtual probe for S. mitis group bacteria that can distinguish between S. pneumoniae and S. mitis/S. oralis was designed:

S. mitis sub-group=(Spar-205p AND AllStrep-261p) OR (Smit-79p AND AllStrep-261p AND NOT Shyo-193p) OR (Ssang-193p AND AllStrep-261p AND NOT Stmu-86p) OR (Stango-85p AND NOT Sang-156p AND AllStrep-261p>0.01) AND IF Smit-79p THEN (Spneu-229 bp/AllStrep-261p)/AllStrep-261p: s3.

### 7.5. Example 5: Virtual Probe for Detecting Species from Enterobacteriaceae Group

Enterobacteriaceae is a large family of gram negative bacteria that includes both pathogenic and non-pathogenic species. Pathogenic family members include Klebsiella species, Enterobacter species, Escherichia species, Citrobacter species, Serratia species and Salmonella species.

The 16s rRNA region for members of the family has minimal genetic sequence variation among the species making it difficult to design 16S probes capable of distinguishing between species. However, given the similarity of 16S sequences, a common probe, “Entb-132p,” was designed which can identify most family members as Enterobacteriace, except Pantoea species which can be identified the probe “Entb-299p.”

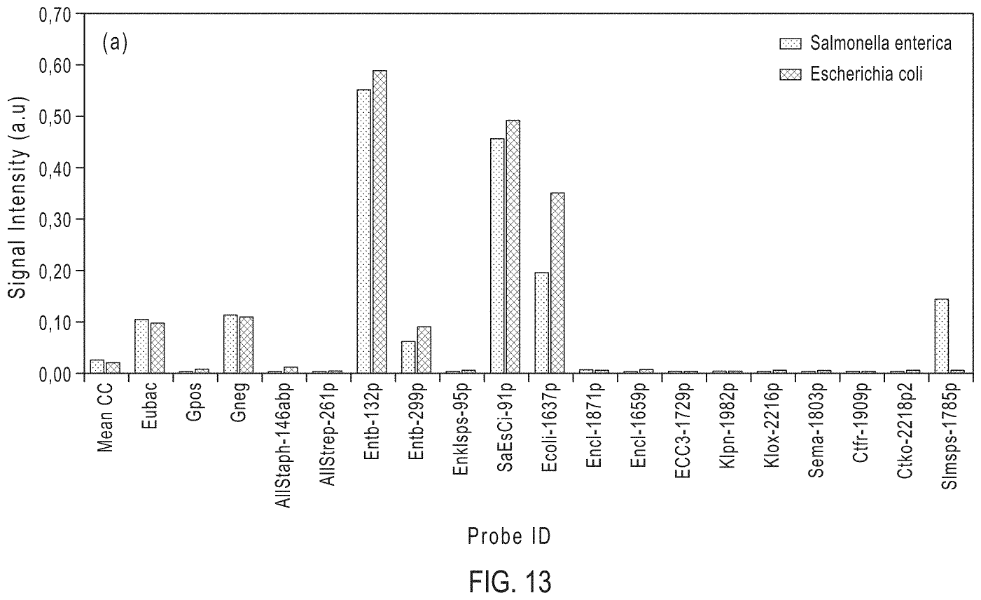

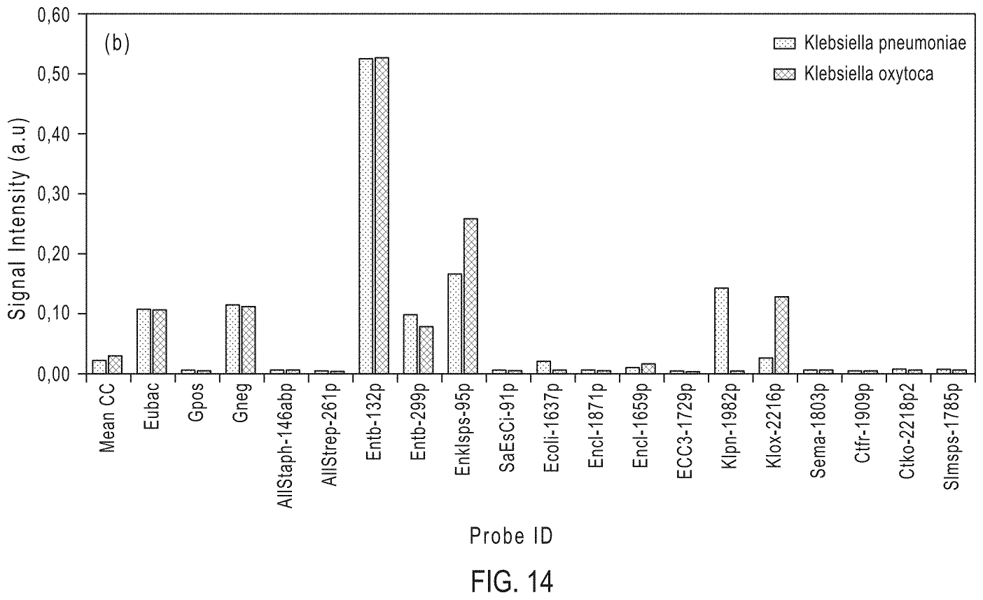

Two group probes were designed following the clustering pattern of the species from Enterobacteriace species in the 16S rRNA genomic region. Species from Enterobacter and Klebsiella genera can be identified by the probe “Enklspss-95p” and species from Citrobacter, Salmonella and Escherichia genera can be identified by the probe “SaEsCi-91p.” Because of the difficulty in designing single probes capable of distinguishing between Enterobacteriaceae species, a combination of 16S rRNA and ITS probes were designed (see FIG. 13 and FIG. 14) for hierarchical identification and differentiation of Enterobacteriaceae species.

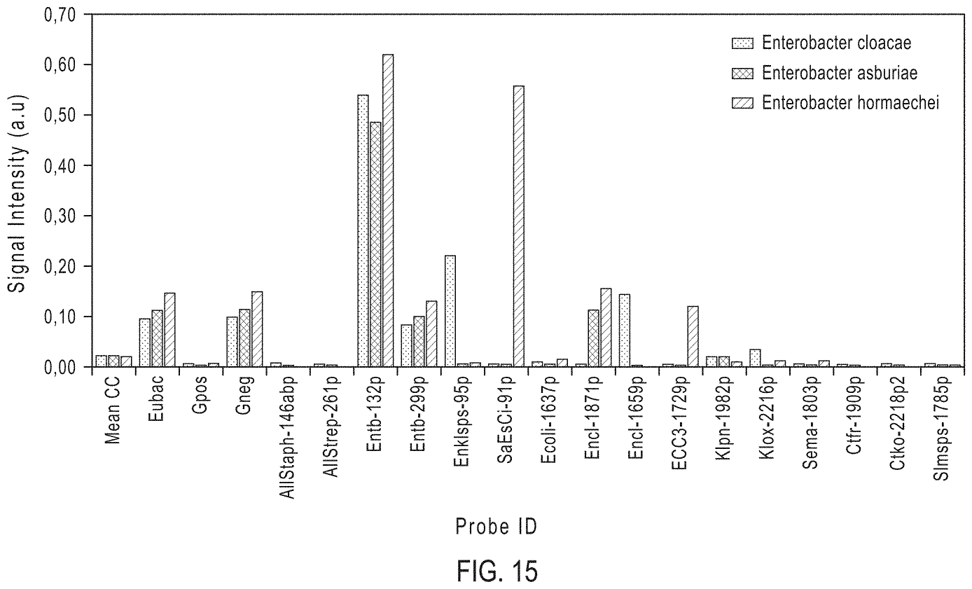

The use of 16s-23s ITS region made it possible to differentiate the species of Enterobacter cloacae complex, which includes E. cloacae, E. asburiae, and E. hormaecheis. Three probes, “Encl-1871p,” “Encl-1659p” and “ECC3-1729p” used in combination allowed for specific identification of different Enterobacter cloacae complex species (see FIG. 15).

E. cloacae=Encl-1659p NOT (Encl-1871p OR ECC3-1729p)
E. asburiae=Enc101871p NOT (Encl-1659p OR ECC3-1729p)
E. hormaechei=(Encl-1871p AND ECC3-1729p) NOT Encl-1659p

### 8. SPECIFIC EMBODIMENTS

The present disclosure is exemplified by the specific embodiments below.

1. A method of determining if a first organism having a first genome or a second organism having a second genome is present in a test sample or an initial sample from which the test sample was prepared, comprising:
(a) probing the test sample with a virtual probe comprising two or more probe molecules, wherein each probe molecule is capable of specifically hybridizing to one or more target nucleic acids corresponding to the first genome and/or one or more homologous target nucleic acids corresponding to the second genome, and wherein the probe molecules hybridize non-identically to the target nucleic acids corresponding to the first and second genomes, such that the hybridizing of the probe molecules to the one or more target nucleic acids corresponding to the first genome and the one or more target nucleic acids corresponding to the second genome can distinguish between the target nucleic acids corresponding to the first genome and the target nucleic acids corresponding to the second genome; and
(b) detecting and/or quantifying signals from hybridization of the probe molecules in the virtual probe to nucleic acids, if any, in the test sample, thereby determining if the first organism or second organism is present in the test sample or initial sample.

2. The method of embodiment 1, wherein the one or more target nucleic acids corresponding to the first genome are a first amplicon set and the one or more target nucleic acids corresponding to the second genome are a second amplicon set, and wherein each probe molecule in the virtual probe is capable of specifically hybridizing to one or more amplicons in the first amplicon set and/or the second amplicon set, and wherein the probe molecules hybridize non-identically to the amplicons in the first amplicon set and the amplicons in the second amplicon set, such that the hybridizing of the probe molecules to the amplicons in the first amplicon set and the second amplicon set can distinguish between the first amplicon set and the second amplicon set.

3. The method of embodiment 2, which further comprises preparing the test sample by performing a PCR amplification reaction on the initial sample using PCR primers capable of hybridizing to, and initiating a PCR amplification from, both the first genome and the second genome, resulting in the first amplicon set and a second amplicon set, respectively, when the first genome and second genome are present in the initial sample.

4. The method of embodiment 3, wherein the PCR primers comprise more than one primer pair and wherein the first amplicon set comprises a plurality of first amplicons and/or the second amplicon set comprises a plurality of second amplicons.

5. The method of embodiment 2, which further comprises preparing the test sample by (a) performing a first PCR amplification reaction on the initial sample using a first set of PCR primers capable of hybridizing to, and initiating a PCR amplification from, both the first genome and the second genome, (b) performing a second PCR amplification reaction on the initial sample using a second set of PCR primers which is different from the first set of PCR primers and which is capable of hybridizing to, and initiating a PCR amplification from, both the first genome and the second genome, and (c) combining the amplicons produced in the first and second PCR reactions, resulting in a first amplicon set comprising a plurality of first amplicons and a second amplicon set comprising a plurality of second amplicons, respectively, when the first genome and second genome are present in the initial sample.

6. The method of embodiment 4 or embodiment 5, wherein the plurality of first amplicons corresponds to different regions in the first genome and/or the plurality of second amplicons corresponds to different regions in the second genome.

7. The method of embodiment 3, wherein the PCR primers comprise a single primer pair and the first amplicon set consists of a single first amplicon and the second amplicon set consists of a single second amplicon.

8. The method of embodiment 7, wherein the nucleotide sequence of the first amplicon and the nucleotide sequence of the second amplicon have at least 1 nucleotide mismatch in the regions of the amplicons capable of hybridizing to at least one probe molecule in the virtual probe.

9. The method of embodiment 7, wherein the nucleotide sequence of the first amplicon and the nucleotide sequence of the second amplicon have at least 2 nucleotide mismatches in the regions of the amplicons capable of hybridizing to at least one probe molecule in the virtual probe.

10. The method of embodiment 7, wherein the nucleotide sequence of the first amplicon and the nucleotide sequence of the second amplicon have at least 3 nucleotide mismatches in the regions of the amplicons capable of hybridizing to at least one probe molecule in the virtual probe.

11. The method of any one of embodiments 3 to 10, wherein the PCR amplification reaction incorporates a label which produces a measurable signal into any amplicons produced by the reaction.

12. The method of any one of embodiments 3 to 11, wherein the primers are labeled.

13. The method of embodiment 12, wherein at least one primer is 5′ fluorescently labeled.

14. The method of embodiment 12, wherein more than one primer is 5′ fluorescently labeled.

15. The method of any one of embodiments 3 to 14, wherein the PCR reaction includes fluorescently labeled deoxynucleotides.

16. The method of any one of embodiments 1 to 15, wherein each probe molecule comprises a nucleotide sequence that is 90% to 100% complementary to 15 to 40 consecutive nucleotides in the first genome and/or second genome.

17. The method of any one of embodiments 1 to 16, wherein the virtual probe comprises two probe molecules having 1 or more nucleotide mismatches relative to one another.

18. The method of embodiment 17, wherein the virtual probe comprises two probe molecules having 1 nucleotide mismatch relative to one another.

19. The method of embodiment 17, wherein the virtual probe comprises probe molecules having 2 nucleotide mismatches relative to one another.

20. The method of any one of embodiments 1 to 19, wherein the probe molecules of the virtual probe are positionally addressable probe molecules present on an array, each at a discrete location on the array.

21. The method of embodiment 20, wherein detecting and/or quantifying signals from hybridization of the probe molecules in the virtual probe to the PCR amplification products comprises detecting and/or quantifying the label at the locations of the probe molecules in the virtual probe.

22. The method of embodiment 20 or embodiment 21, wherein step (b) comprises:
(i) contacting the PCR amplification products with the array;
(ii) washing unbound nucleic acid molecules from the array; and
(iii) measuring the signal intensity of the label at each probe molecule location on the array.

23. The method of any one of embodiments 20 to 22, wherein the array comprises one or more control probe molecules.

24. The method of any one of embodiments 20 to 23, wherein the probe molecules are oligonucleotide probe molecules.

25. The method of embodiment 24, wherein one or more of the probe molecules have a poly-thymidine tail.

26. The method of embodiment 24, wherein the poly-thymidine tail is a 10-mer to a 20-mer.

27. The method of embodiment 26, wherein the poly-thymidine tail is a 15-mer.

28. The method of any one of embodiments 3 to 19, the PCR amplification reaction is a real-time PCR amplification reaction.

29. The method of embodiment 28, wherein:
(a) each probe molecule comprises a distinguishable label and a quencher moiety that inhibits detection of the label when the label and quencher moiety are both attached to the probe;
(b) the label produces a measurable signal upon cleavage of the probe molecule during the real-time PCR amplification reaction; and
(c) each label is distinguishable from each other label.

30. The method of embodiment 29, wherein the labels are fluorescent labels.

31. The method of any one of embodiments 2 to 30, wherein the first amplicon set and the second amplicon set each comprise a nucleotide sequence corresponding to a gene encoding rRNA.

32. The method of any one of embodiments 2 to 31, wherein the first amplicon set and the second amplicon set each comprise a nucleotide sequence corresponding to an intergenic spacer region between rRNA genes.

33. The method of any one of embodiments 1 to 32, wherein the first organism and second organism are microorganisms.

34. The method of embodiment 33, wherein the microorganisms are members of the same order.

35. The method of embodiment 33, wherein the microorganisms are members of the same family.

36. The method of embodiment 35, wherein the microorganisms are members of the same genus.

37. The method of embodiment 36, wherein the microorganisms are members of the same group.

38. The method of any one of embodiments 33 to 37, wherein one or more of the microorganisms is a human pathogen or an animal pathogen.

39. The method of any one of embodiments 33 to 38, wherein the microorganisms are bacteria, viruses, or fungi.

40. The method of any one of embodiments 33 to 39, wherein the microorganisms are bacteria.

41. The method of embodiment 40, wherein the first amplicon set and the second amplicon set each comprise a nucleotide sequence corresponding to a 16S rRNA gene and/or a nucleotide sequence corresponding to a 23S rRNA gene.

42. The method of embodiment 41, wherein the first amplicon set and the second amplicon set each comprise a nucleotide sequence corresponding to a 16S rRNA gene.

43. The method of embodiment 41 or embodiment 42, wherein the first amplicon set and the second amplicon set each comprise a nucleotide sequence corresponding to a 23S rRNA gene.

44. The method of any one of embodiments 40 to 43, wherein the first amplicon set and the second amplicon set each comprise a nucleotide sequence corresponding to a 16S-23S intergenic spacer region.

45. The method any one of embodiments 1 to 44, wherein the signals from the hybridization of the probe molecules to target nucleic acids are combinable by (i) one or more Boolean operators, (ii) one or more relational operators, or (iii) one or more Boolean operators and one or more relational operators to distinguish between the first genome and the second genome.

46. The method of embodiment 45, which further comprises combining the signals from the hybridization of the probe molecules in the virtual probe to the target nucleic acids by (i) one or more Boolean operators, (ii) one or more relational operators, or (iii) one or more Boolean operators and one or more relational operators to distinguish between the first genome and the second genome.

47. The method of embodiment 45 or embodiment 46, wherein each Boolean operator is independently selected from “AND”, “OR”, and “NOT”.

48. The method of any one of embodiments 45 to 47, wherein each relational operator is independently selected from “greater than” (“>”) and “less than” (“<”).

49. The method of any one of embodiments 45 to 47, wherein the signals are combinable by one or more Boolean operators.

50. The method of any one of embodiments 45 to 48, wherein the signals are combinable by one or more relational operators.

51. The method of any one of embodiments 45 to 48, wherein the signals are combinable by one or more Boolean operators and one or more relational operators.

52. The method of any one of embodiments 1 to 51, wherein the virtual probe comprises or consists of two probe molecules.

53. The method of embodiment 52, wherein the virtual probe comprises (i) a first probe molecule capable of specifically hybridizing to a first target nucleic acid (e.g., a first amplicon in the first amplicon set when the target nucleic acids are PCR products) and a second target nucleic acid (e.g., a second amplicon in the second amplicon set when the target nucleic acids are PCR products) and (ii) a second probe molecule that is capable of specifically hybridizing to the second target nucleic acid but not the first target nucleic acid.

54. The method of embodiment 53, which comprises determining that the first organism is present in the test sample or initial sample if the signal for the first probe molecule is positive and the signal for the second probe molecule is not positive.

55. The method of embodiment 53 or embodiment 54, which comprises determining that the second organism is present in the test sample or initial sample if the signal for the first probe molecule is positive and the signal for the second probe molecule is positive.

56. The method of any one of embodiments 53 to 55, wherein the first microorganism is a coagulase negative Staphylococcus sp. and the second microorganism is a coagulase positive Staphylococcus sp.

57. The method of embodiment 56, wherein the second microorganism is S. aureus.

58. The method of any one of embodiment 56 or embodiment 57, wherein the first probe molecule has a nucleotide sequence comprising CCAGTCTTATAGGTAGGTTAYCCACG (SEQ ID NO:1).

59. The method of any one of embodiments 56 to embodiment 58, wherein the second probe molecule has a nucleotide sequence comprising GCTTCTCGTCCGTTCGCTCG (SEQ ID NO:2).

60. The method of any one of embodiments 53 to 55, wherein the first microorganism is Streptococcus gordonii and the second microorganism is Streptococcus anginosus.

61. The method of embodiment 60, wherein the first probe molecule has a nucleotide sequence comprising CAGTCTATGGTGTAGCAAGCTACGGTAT (SEQ ID NO:3).

62. The method of embodiment 60 or embodiment 61, wherein the second probe molecule has a nucleotide sequence comprising TATCCCCCTCTAATAGGCAGGTTA (SEQ ID NO:4).

63. The method of any one of embodiments 53 to 55, wherein the first and second microorganisms are Enterobacteriaceae bacteria.

64. The method of embodiment 63, wherein the first and second microorganisms are selected from Enterobacter aerogenes, Enterobacter asburiae, and Enterobacter hormaechei.

65. The method of embodiment 52, wherein the virtual probe comprises (i) a first probe molecule capable of specifically hybridizing to a first target nucleic acid (e.g., a first amplicon in the first amplicon set when the target nucleic acids are PCR products) and a second target nucleic acid (e.g., a second amplicon in the second amplicon set when the target nucleic acids are PCR products) and (ii) a second probe molecule that is capable of specifically hybridizing to the first target nucleic acid and the second target nucleic acid.

66. The method of embodiment 65, which comprises determining that the first organism is present in the test sample or initial sample if the signal for the first probe molecule divided by the signal for the second probe molecule is less than a predetermined cutoff value.

67. The method of embodiment 65 or embodiment 66, which comprises determining that the second organism is present in the test sample or initial sample if the signal for the first probe molecule divided by the signal for the second probe molecule is greater than a predetermined cutoff value.

68. The method of any one of embodiments 65 to 67, wherein the first microorganism is Streptococcus mitis and the second microorganism is Streptococcus pneumoniae

69. The method of any one of embodiments 65 to 68, wherein the first probe molecule has a nucleotide sequence comprising GATGCAAGTGCACCTTTTAAGTAA (SEQ ID NO:7).

70. The method of any one of embodiments 65 to 69, wherein the second probe molecule has a nucleotide sequence comprising AGCTAATACAACGCAGGTCCATCT (SEQ ID NO:5).

71. The method of any one of embodiments 1 to 51, wherein the virtual probe comprises or consists of three probe molecules.

72. The method of embodiment 71, wherein the virtual probe comprises (i) a first probe molecule capable of specifically hybridizing to a first target nucleic acid (e.g., a first amplicon in the first amplicon set when the target nucleic acids are PCR products) and a second target nucleic acid (e.g., a second amplicon in the second amplicon set when the target nucleic acids are PCR products), (ii) a second probe molecule which is different from the first probe molecule and that capable of specifically hybridizing to the first and second target nucleic acids, and (iii) a third probe molecule which is different from the first and second probe molecules and that is capable of specifically hybridizing to the first and second target nucleic acids.

73. The method of embodiment 72, which comprises determining that the first organism is present in the test sample or initial sample if:
(a) the signal for the first probe molecule is positive or the signal for the second probe molecule is positive, and
(b) the signal for the first probe molecule or the signal for the second probe molecule is less than the signal or a proper fraction of the signal for the third probe molecule.

74. The method of embodiment 72 or embodiment 73, which comprises determining that the second organism is present in the test sample or initial sample if:
(a) the signal for the first probe molecule and the signal for the second probe molecule is positive, and
(b) the signal for the first probe molecule and the signal for the second probe molecule is greater than the signal or a proper fraction of the signal for the third probe molecule.

75. The method of any one of embodiments 65 to 74, wherein the first microorganism is Streptococcus mitis and the second microorganism is Streptococcus pneumoniae.

76. The method of embodiment 75, wherein the first probe molecule has a nucleotide sequence comprising GATGCAAGTGCACCTTTTAAGCAA (SEQ ID NO:6).

77. The method of embodiment 75 or embodiment 76, wherein the second probe molecule has a nucleotide sequence comprising GATGCAAGTGCACCTTTTAAGTAA (SEQ ID NO:7).

78. The method of any one of embodiments 75 to 77, wherein the third probe molecule has a nucleotide sequence comprising AGCTAATACAACGCAGGTCCATCT (SEQ ID NO:5).

79. The method of any one of embodiments 3 to 78, wherein the PCR conditions are selected so that the PCR amplification products are 300 to 800 nucleotides in length.

80. The method of embodiment 79, wherein the PCR conditions are selected so that the PCR amplification products are 400 to 600 nucleotides in length.

81. The method of any one of embodiments 33 to 80, wherein the initial sample or test sample is at risk of infection with one or more of the microorganisms.

82. The method of any one of embodiments 33 to 81, wherein the initial sample or test sample is suspected of having an infection with one or more of the microorganisms.

83. The method of any one of embodiments 1 to 82, wherein the initial sample or test sample is a biological sample, an environmental sample, or a food product.

84. The method of embodiment 83, wherein the initial sample or test sample is a biological sample selected from blood, serum, saliva, urine, gastric fluid, digestive fluid, tears, stool, semen, vaginal fluid, interstitial fluid, fluid derived from tumorous tissue, ocular fluid, sweat, mucus, earwax, oil, glandular secretions, breath, spinal fluid, hair, fingernails, skin cells, plasma, fluid obtained from a nasal swab, fluid obtained from a nasopharyngeal wash, cerebrospinal fluid, a tissue sample, fluid or tissue obtained from a throat swab, fluid or tissue obtained from a wound swab, biopsy tissue, placental fluid, amniotic fluid, peritoneal dialysis fluid, cord blood, lymphatic fluids, cavity fluids, sputum, pus, microbiota, meconium, breast milk, or a sample processed, extracted or fractionated from any of the foregoing.

85. The method of embodiment 84, wherein the biological sample is:
(a) urine, sputum or a sample processed, extracted or fractionated from urine;
(b) sputum or a sample processed, extracted or fractionated from sputum;
(c) a wound swab or a sample processed, extracted or fractionated from a wound swab;
(d) blood or a sample processed, extracted or fractionated from blood; or
(e) peritoneal dialysis fluid or a sample processed, extracted or fractionated from peritoneal dialysis fluid.

86. The method of embodiment 83, wherein the initial sample or test sample is an environmental sample selected from soil, groundwater, surface water, wastewater, or a sample processed, extracted or fractionated from any of the foregoing.

87. An addressable array, comprising:
(a) one or more virtual probes for distinguishing a first genomic sequence from a second, homologous genomic sequence, each virtual probe comprising a group of positionally addressable oligonucleotide probe molecules, each at a discrete location on the array, wherein each probe molecule in the one or more virtual probes comprises a nucleotide sequence that is 90% to 100% complementary to 15 to 40 consecutive nucleotides in the first genomic sequence or second genomic sequence; and
(b) optionally, one or more control probe molecules.

88. The addressable array of embodiment 87, which comprises at least two virtual probes.

89. The addressable array of embodiment 87, which comprises at least three virtual probes.

90. The addressable array of embodiment 87, which comprises at least four virtual probes.

91. The addressable array of embodiment 87, which comprises at least five virtual probes.

92. The addressable array of embodiment 87, which comprises at least ten virtual probes.

93. The addressable array of any one of embodiments 87 to 91, which comprises up to ten virtual probes.

94. The addressable array of any one of embodiments 87 to 92, which comprises up to fifteen virtual probes.

95. The addressable array of any one of embodiments 87 to 94, wherein each virtual probe comprises 2-4 oligonucleotide probe molecules.

96. The addressable array of embodiment 95, wherein each virtual probe comprises 2-3 oligonucleotide probe molecules.

97. The addressable array of any one of embodiments 87 to 96, which comprises 12 or more probe molecules.

98. The addressable array of embodiment 97, which comprises 12 to 100 probe molecules.

99. The addressable array of embodiment 97, which comprises 12 to 50 probe molecules.

100. The addressable array of embodiment 97, which comprises 25 to 75 probe molecules.

101. The addressable array of embodiment 97, which comprises 50 to 100 probe molecules.

102. The addressable array of embodiment 97, which comprises 12 probe molecules.

103. The addressable array of embodiment 97, which comprises 14 probe molecules.

104. The addressable array of embodiment 97, which comprises 84 probe molecules.

105. The addressable array of any one of embodiments embodiment 87 to 104, wherein the first genomic sequence and the second genomic sequence are genomic sequences from a first microorganism and a second microorganism, respectively.

106. The addressable array of embodiment 105, wherein the microorganisms are members of the same order.

107. The addressable array of embodiment 105, wherein the microorganisms are members of the same family.

108. The addressable array of embodiment 107, wherein the microorganisms are members of the same genus.

109. The addressable array of embodiment 108, wherein the microorganisms are members of the same group.

110. The addressable array of any one of embodiments 87 to 109, wherein one or more of the probe molecules comprise a poly-thymidine tail.

111. The addressable array of embodiment 110, wherein the poly-thymidine tail is a 10-mer to a 20-mer.

112. The addressable array of embodiment 111, wherein the poly-thymidine tail is a 15-mer.

113. The addressable array of any one of embodiments 87 to 112, wherein the first genomic sequence and the second genomic sequence each comprise a nucleotide sequence corresponding to a gene encoding rRNA.

114. The addressable array of embodiment 113, wherein the gene encoding rRNA is a 16S rRNA gene or a 23SrRNA gene.

115. The addressable array of any one of embodiments 87 to 112, wherein the first genomic sequence and the second genomic sequence each comprise a nucleotide sequence corresponding to an intergenic spacer region between rRNA genes.

116. The addressable array of any one of embodiments 87 to 115, in which at least one virtual probe comprises probe molecules for differentiating a genomic sequence from a species of eubacteria from a genomic sequence of a microorganism which is not a species of eubacteria.

117. The addressable array of any one of embodiments 87 to 116, in which at least one virtual probe comprises probe molecules for differentiating a genomic sequence from a gram positive bacteria and a genomic sequence from a gram negative bacteria.

118. The addressable array of any one of embodiments 87 to 117, in which at least one virtual probe comprises probe molecules for differentiating genomic sequences from microorganisms of different orders.

119. The addressable array of any one of embodiments 87 to 118, in which at least one virtual probe comprises probe molecules for differentiating genomic sequences from microorganisms of different families.

120. The addressable array of any one of embodiments 87 to 119, in which at least one virtual probe comprises probe molecules for differentiating genomic sequences from microorganisms of different genera.

121. The addressable array of any one of embodiments 87 to 120, in which at least one virtual probe comprises probe molecules for differentiating genomic sequences from microorganisms of different groups.

122. The addressable array of any one of embodiments 87 to 121, in which at least one virtual probe comprises probe molecules for differentiating genomic sequences from microorganisms of different species.

123. The addressable array of any one of embodiments 87 to 122, in which at least one virtual probe comprises a probe molecule whose nucleotide sequence comprises CCAGTCTTATAGGTAGGTTAYCCACG (SEQ ID NO:1).

124. The addressable array of any one of embodiments 87 to 123, in which at least one virtual probe comprises a probe molecule whose nucleotide sequence comprises GCTTCTCGTCCGTTCGCTCG (SEQ ID NO:2).

125. The addressable array of any one of embodiments 87 to 124, in which at least one virtual probe comprises a probe molecule whose nucleotide sequence comprises CAGTCTATGGTGTAGCAAGCTACGGTAT (SEQ ID NO:3).

126. The addressable array of any one of embodiments 87 to 125, in which at least one virtual probe comprises a probe molecule whose nucleotide sequence comprises TATCCCCCTCTAATAGGCAGGTTA (SEQ ID NO:4).

127. The addressable array of any one of embodiments 87 to 126, in which at least one virtual probe comprises a probe molecule whose nucleotide sequence comprises AGCTAATACAACGCAGGTCCATCT (SEQ ID NO:5).

128. The addressable array of any one of embodiments 87 to 127, in which at least one virtual probe comprises a probe molecule whose nucleotide sequence comprises GATGCAAGTGCACCTTTTAAGCAA (SEQ ID NO:6).

129. The addressable array of any one of embodiments 87 to 128, in which at least one virtual probe comprises a probe molecule whose nucleotide sequence comprises GATGCAAGTGCACCTTTTAAGTAA (SEQ ID NO:7).

130. A method of determining if a first organism having a first genome or a second organism having a second genome is present in a test sample or an initial sample from which the test sample is derived, comprising:
(a) probing the test sample with an array according to any one of embodiments 87 to 129 which comprises a virtual probe comprising two or more probe molecules, wherein each probe molecule is capable of specifically hybridizing to one or more target nucleic acids corresponding to the first genome and/or one or more homologous target nucleic acids corresponding to the second genome, and wherein the probe molecules hybridize non-identically to the target nucleic acids corresponding to the first and second genomes, such that the hybridizing of the probe molecules to the one or more target nucleic acids corresponding to the first genome and the one or more target nucleic acids corresponding to the second genome can distinguish between the target nucleic acids corresponding to the first genome and the target nucleic acids corresponding to the second genome; and
(b) washing unbound nucleic acid molecules from the array;
(c) detecting and/or quantifying the signal at each probe molecule location on the array; and
(d) if the signals indicate that:
   (i) target nucleic acids that hybridize to the probe molecules of the array are present in the test sample, analyzing the signals to determine if target nucleic acids corresponding to the first genome or target nucleic acids corresponding to the second genome are present in the sample, thereby determining if the first organism or second organism are present in the initial sample or the test sample; or
   (ii) no target products that hybridize to the probe molecules of the virtual probe are produced in step (a), determining that that initial sample or test sample does not contain the first organism or the second organism,
thereby determining if the first organism or second organism is present in the initial sample or the test sample.

131. The method of embodiment 130, wherein the one or more target nucleic acids corresponding to the first genome are a first amplicon set and the one or more target nucleic acids corresponding to the second genome are a second amplicon set, and wherein each probe molecule in the virtual probe is capable of specifically hybridizing to one or more amplicons in the first amplicon set and/or the second amplicon set, and wherein the probe molecules hybridize non-identically to the amplicons in the first amplicon set and the amplicons in the second amplicon set, such that the hybridizing of the probe molecules to the amplicons in the first amplicon set and the second amplicon set can distinguish between the first amplicon set and the second amplicon set.

132. The method of embodiment 131, which further comprises preparing the test sample by performing a PCR amplification reaction on the initial sample using PCR primers capable of hybridizing to, and initiating a PCR amplification from, both the first genome and the second genome, resulting in the first amplicon set and a second amplicon set, respectively, when the first genome and second genome are present in the sample.

133. A system for determining if an organism is present in a sample, comprising:
(a) an optical reader for generating signal data for each probe molecule location of the array of any one of embodiments 87 to 129; and
(b) at least one processor which:
   (i) is configured to receive signal data from the optical reader;
   (ii) is configured to analyze the signal data for the one or more virtual probes; and
   (iii) has an interface to a storage or display device or network for outputting a result of the analysis.

134. The system of embodiment 133, further comprising a plate handling robot capable of adding the product of a PCR amplification reaction to the array and capable of washing unbound nucleic acid molecules from the array.

135. The method of any one of embodiments 1 to 86 or 130 to 132, which is performed using the system of embodiment 133 or 134.

136. An oligonucleotide probe molecule whose nucleotide sequence comprises CCAGTCTTATAGGTAGGTTAYCCACG (SEQ ID NO:1).

137. An oligonucleotide probe molecule whose nucleotide sequence comprises GCTTCTCGTCCGTTCGCTCG (SEQ ID NO:2).

138. An oligonucleotide probe molecule whose nucleotide sequence comprises CAGTCTATGGTGTAGCAAGCTACGGTAT (SEQ ID NO:3).

139. An oligonucleotide probe molecule whose nucleotide sequence comprises TATCCCCCTCTAATAGGCAGGTTA (SEQ ID NO:4).

140. An oligonucleotide probe molecule whose nucleotide sequence comprises AGCTAATACAACGCAGGTCCATCT (SEQ ID NO:5).

141. An oligonucleotide probe molecule whose nucleotide sequence comprises GATGCAAGTGCACCTTTTAAGCAA (SEQ ID NO:6).

142. An oligonucleotide probe molecule whose nucleotide sequence comprises GATGCAAGTGCACCTTTTAAGTAA (SEQ ID NO:7).

143. The oligonucleotide probe molecule of any one of embodiments 136 to 142, which comprises a poly-thymidine tail.

144. The oligonucleotide probe molecule of embodiment 143, wherein the poly-thymidine tail is a 10-mer to a 20-mer.

145. The oligonucleotide probe molecule of embodiment 144, wherein the poly-thymidine tail is a 15-mer.

146. The oligonucleotide probe molecule of any one of embodiments 136 to 145, which comprises a label.

147. A virtual probe comprising a plurality of oligonucleotide probe molecules, wherein at least one oligonucleotide probe molecule in the virtual probe has a nucleotide sequence comprising CCAGTCTTATAGGTAGGTTAYCCACG (SEQ ID NO:1) and another oligonucleotide molecule in the virtual probe has a nucleotide sequence comprising GCTTCTCGTCCGTTCGCTCG (SEQ ID NO:2).

148. A virtual probe comprising a plurality of oligonucleotide probe molecules, wherein at least one oligonucleotide probe molecule in the virtual probe has a nucleotide sequence comprising CAGTCTATGGTGTAGCAAGCTACGGTAT (SEQ ID NO:3) and another oligonucleotide molecule in the virtual probe has a nucleotide sequence comprising TATCCCCCTCTAATAGGCAGGTTA (SEQ ID NO:4).

149. A virtual probe comprising a plurality of oligonucleotide probe molecules, wherein at least one oligonucleotide probe molecule in the virtual probe has a nucleotide sequence comprising AGCTAATACAACGCAGGTCCATCT (SEQ ID NO:5), another oligonucleotide molecule in the virtual probe has a nucleotide sequence comprising GATGCAAGTGCACCTTTTAAGCAA (SEQ ID NO:6) and another oligonucleotide molecule in the virtual probe has a nucleotide sequence comprising GATGCAAGTGCACCTTTTAAGTAA (SEQ ID NO:7).

150. The virtual probe of any one of embodiments 147 to 149, in which each oligonucleotide probe molecule comprises a poly-thymidine tail.

151. The virtual probe of embodiment 150, wherein the poly-thymidine tail is a 10-mer to a 20-mer.

152. The virtual probe of embodiment 151, wherein the poly-thymidine tail is a 15-mer.

153. An addressable array comprising:
(a) a group of positionally addressable probe molecules, each at a discrete location on the array, wherein the group of probe molecules comprises the oligonucleotide probe molecule of any one of embodiments 136 to 146; and
(b) optionally, one or more control probe molecules.

154. An addressable array comprising the virtual probe of any one of embodiments 147 to 152, wherein each probe molecule in the virtual probe is at a discrete location in the array.

155. The addressable array of embodiment 154, which further comprises one or more control probe molecules.

156. A kit comprising two or more probe molecules selected from probe molecules whose nucleotide sequence comprises SEQ ID NO:1, SEQ ID NO:2, SEQ ID NO:3, SEQ ID NO:4, SEQ ID NO:5, SEQ ID NO:6, or SEQ ID NO:7.

157. The kit of embodiment 156, which comprises an oligonucleotide probe molecule whose nucleotide sequence comprises SEQ ID NO:1 and an oligonucleotide probe molecule whose nucleotide sequence comprises SEQ ID NO:2.

158. The kit of embodiment 156, which comprises an oligonucleotide probe molecule whose nucleotide sequence comprises SEQ ID NO:3 and an oligonucleotide probe molecule whose nucleotide sequence comprises SEQ ID NO:4.

159. The kit of embodiment 156, which comprises an oligonucleotide probe molecule whose nucleotide sequence comprises SEQ ID NO:5, an oligonucleotide probe molecule whose nucleotide sequence comprises SEQ ID NO:6, and an oligonucleotide probe molecule whose nucleotide sequence comprises SEQ ID NO:7.

160. The kit of embodiment 156, which comprises an oligonucleotide probe molecule whose nucleotide sequence comprises SEQ ID NO:1, an oligonucleotide probe molecule whose nucleotide sequence comprises SEQ ID NO:2, an oligonucleotide probe molecule whose nucleotide sequence comprises SEQ ID NO:3 and an oligonucleotide probe molecule whose nucleotide sequence comprises SEQ ID NO:4.

161. The kit of embodiment 156, which comprises an oligonucleotide probe molecule whose nucleotide sequence comprises SEQ ID NO:1, an oligonucleotide probe molecule whose nucleotide sequence comprises SEQ ID NO:2, an oligonucleotide probe molecule whose nucleotide sequence comprises SEQ ID NO:5, an oligonucleotide probe molecule whose nucleotide sequence comprises SEQ ID NO:6, and an oligonucleotide probe molecule whose nucleotide sequence comprises SEQ ID NO:7.

162. The kit of embodiment 156, which comprises an oligonucleotide probe molecule whose nucleotide sequence comprises SEQ ID NO:3, an oligonucleotide probe molecule whose nucleotide sequence comprises SEQ ID NO:4, an oligonucleotide probe molecule whose nucleotide sequence comprises SEQ ID NO:5, an oligonucleotide probe molecule whose nucleotide sequence comprises SEQ ID NO:6, and an oligonucleotide probe molecule whose nucleotide sequence comprises SEQ ID NO:7.

163. The kit of embodiment 156, which comprises an oligonucleotide probe molecule whose nucleotide sequence comprises SEQ ID NO:1, an oligonucleotide probe molecule whose nucleotide sequence comprises SEQ ID NO:2, an oligonucleotide probe molecule whose nucleotide sequence comprises SEQ ID NO:3, an oligonucleotide probe molecule whose nucleotide sequence comprises SEQ ID NO:4 an oligonucleotide probe molecule whose nucleotide sequence comprises SEQ ID NO:5, an oligonucleotide probe molecule whose nucleotide sequence comprises SEQ ID NO:6, and an oligonucleotide probe molecule whose nucleotide sequence comprises SEQ ID NO:7.

164. The kit of any one of embodiments 156 to 163, wherein the probe molecules are labeled.

165. The kit of embodiment 164, wherein the probe molecules are labeled with a fluorescent label.

166. The kit of any one of embodiments 156 to 163, wherein the probe molecules are unlabeled.

167. The kit of any one of embodiments 156 to 166, which further comprises one or more PCR primer pairs capable of amplifying a first genomic sequence, and a second, homologous genomic sequence.

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. A method of detecting if a first organism having a first genome or a second organism having a second genome is present in a test sample or an initial sample from which the test sample was prepared, wherein the first and second organisms are Staphylococci, the method comprising:
(a) probing the test sample with a virtual probe comprising a plurality of probe molecules, wherein
(i) the virtual probe comprises at least two probe molecules is capable of specifically hybridizing to one or more target nucleic acids corresponding to a 16S rRNA gene of the first genome and/or the second genome,
(ii) the virtual probe optionally further comprises one or more probe molecules capable of specifically hybridizing to one or more target nucleic acids corresponding to a 16S-23S intergenic spacer region of the first genome and/or the second genome;
(iii) the probe molecules cannot individually distinguish the target nucleic acids corresponding to the first and second genomes, and
(iv) the probe molecules hybridize non-identically to the target nucleic acids corresponding to the first and second genomes, such that the hybridizing of the probe molecules to the target nucleic acids corresponding to the first and second genomes can distinguish between the target nucleic acids corresponding to the first and second genomes; and
(b) detecting and/or quantifying signals from hybridization of the probe molecules in the virtual probe to nucleic acids, if any, in the test sample.

2. The method of claim 1, wherein the one or more target nucleic acids corresponding to the first genome are a first amplicon set and the one or more target nucleic acids corresponding to the second genome are a second amplicon set, and wherein each probe molecule in the virtual probe is capable of specifically hybridizing to one or more amplicons in the first amplicon set and/or the second amplicon set, and wherein the probe molecules hybridize non-identically to the amplicons in the first amplicon set and the amplicons in the second amplicon set, such that the hybridizing of the probe molecules to the amplicons in the first amplicon set and the second amplicon set can distinguish between the first amplicon set and the second amplicon set.

3. The method of claim 2, which further comprises preparing the test sample by performing a PCR amplification reaction on the initial sample using PCR primers capable of hybridizing to, and initiating a PCR amplification from, both the first genome and the second genome, resulting in the first amplicon set and a second amplicon set, respectively, when the first genome and second genome are present in the initial sample.

4. The method of claim 3, wherein the PCR primers comprise more than one primer pair and wherein the first amplicon set comprises a plurality of first amplicons and/or the second amplicon set comprises a plurality of second amplicons.

5. The method of claim 4, wherein the plurality of first amplicons comprises nucleotide sequences corresponding to a portion of the 16S rRNA gene and the 16S-23S intergenic spacer region of to the first genome and/or the plurality of second amplicons comprises nucleotide sequences corresponding to a portion of the 16S rRNA gene and the 16S-23S intergenic spacer region of the second genome.

6. The method of claim 3, wherein the PCR primers comprise a single primer pair and the first amplicon set consists of a single first amplicon corresponding to a portion of the 16S rRNA gene of the first genome and the second amplicon set consists of a single second amplicon corresponding to a portion of the 16S rRNA gene of the second genome.

7. The method of claim 1, wherein the probe molecules of the virtual probe are positionally addressable probe molecules present on an array, each at a discrete location on the array.

8-25. (canceled)

26. The method of claim 1, wherein the first microorganism is a coagulase negative Staphylococcus sp. and the second microorganism is a coagulase positive Staphylococcus sp.

27. The method of claim 26, wherein at least one probe molecule in the virtual probe has a nucleotide sequence comprising CCAGTCTTATAGGTAGGTTAYCCACG (SEQ ID NO:1) and another probe molecule in the virtual probe has a nucleotide sequence comprising GCTTCTCGTCCGTTCGCTCG (SEQ ID NO:2).

28. The method of claim 5, wherein the first microorganism is a coagulase negative Staphylococcus sp. and the second microorganism is a coagulase positive Staphylococcus sp.

29. The method of claim 28, wherein at least one probe molecule in the virtual probe has a nucleotide sequence comprising CCAGTCTTATAGGTAGGTTAYCCACG (SEQ ID NO:1) and another probe molecule in the virtual probe has a nucleotide sequence comprising GCTTCTCGTCCGTTCGCTCG (SEQ ID NO:2).

30. The method of claim 6, wherein the first microorganism is a coagulase negative Staphylococcus sp. and the second microorganism is a coagulase positive Staphylococcus sp.

31. The method of claim 30, wherein at least one probe molecule in the virtual probe has a nucleotide sequence comprising CCAGTCTTATAGGTAGGTTAYCCACG (SEQ ID NO:1) and another probe molecule in the virtual probe has a nucleotide sequence comprising GCTTCTCGTCCGTTCGCTCG (SEQ ID NO:2).

32. The method of claim 1, wherein the initial sample or test sample is a biological sample, an environmental sample, or a food product.

33. The method of claim 32, wherein the initial sample or test sample is a biological sample selected from blood, serum, saliva, urine, gastric fluid, digestive fluid, tears, stool, semen, vaginal fluid, interstitial fluid, fluid derived from tumorous tissue, ocular fluid, sweat, mucus, earwax, oil, glandular secretions, breath, spinal fluid, hair, fingernails, skin cells, plasma, fluid obtained from a nasal swab, fluid obtained from a nasopharyngeal wash, cerebrospinal fluid, a tissue sample, fluid or tissue obtained from a throat swab, fluid or tissue obtained from a wound swab, biopsy tissue, placental fluid, amniotic fluid, peritoneal dialysis fluid, cord blood, lymphatic fluids, cavity fluids, sputum, pus, microbiota, meconium, breast milk, or a sample processed, extracted or fractionated from any of the foregoing.

34. The method of claim 1, wherein the initial sample or test sample is:
(a) urine, sputum or a sample processed, extracted or fractionated from urine;
(b) sputum or a sample processed, extracted or fractionated from sputum;
(c) a wound swab or a sample processed, extracted or fractionated from a wound swab;
(d) blood or a sample processed, extracted or fractionated from blood; or
(e) peritoneal dialysis fluid or a sample processed, extracted or fractionated from peritoneal dialysis fluid.


SEQUENCE LISTING (7 sequences)

SEQ ID NO: 1 - DNA, 26 bp, Artificial Sequence
  AllStaph-146abp
ccagtcttataggtaggttayccacg

SEQ ID NO: 2 - DNA, 20 bp, Artificial Sequence
  Sau-71p
gcttctcgtccgttcgctcg

SEQ ID NO: 3 - DNA, 28 bp, Artificial Sequence
  Stango 85p
cagtctatggtgtagcaagctacggtat

SEQ ID NO: 4 - DNA, 24 bp, Artificial Sequence
  Sang 156p
tatccccctctaataggcaggtta

SEQ ID NO: 5 - DNA, 24 bp, Artificial Sequence
  AllStrep-261p
agctaatacaacgcaggtccatct

SEQ ID NO: 6 - DNA, 24 bp, Artificial Sequence
  Spneu-229p
gatgcaagtgcaccttttaagcaa

SEQ ID NO: 7 - DNA, 24 bp, Artificial Sequence
  Spneu-229bp
gatgcaagtgcaccttttaagtaa